(19) United States
(12) Patent Application Publication
(10) Pub. No.: US 2022/0081389 A1
(43) Pub. Date: Mar. 17, 2022

(54) AMIDE COMPOUNDS AND PREPARATION METHOD THEREFOR AND USE THEREOF

(71) Applicant: CAC NANTONG CHEMICAL CO., LTD., Jiangsu (CN)

(72) Inventors: Jiyong LIU, Shanghai (CN); Liang LV, Shanghai (CN); Liqi ZHOU, Shanghai (CN); Yonglei DU, Shanghai (CN); Juncheng XIANG, Shanghai (CN); Jueping NI, Shanghai (CN)

(21) Appl. No.: 17/424,594

(22) PCT Filed: Sep. 22, 2020
(86) PCT No.: PCT/CN2020/116841

§ 371 Date: Jul. 21, 2021

(30) Foreign Application Priority Data
Oct. 15, 2019 (CN) 201910978806.0
Oct. 15, 2019 (CN) 201910979827.4
Sep. 16, 2020 (CN) 202010973200.0

(51) Int. Cl. C07C 233/75; C07D 213/81; A01N 37/18; A01N 43/40

(52) U.S. Cl. CPC C07C 233/75; A01N 43/40; A01N 37/18; C07D 213/81

(57) ABSTRACT

Provided are amide compounds and a preparation method therefor and the use thereof. The amide compounds have a structure represented by formula I. The amide compounds of the present invention have high insecticidal activity at a low dosage and have a good fast-acting property. The dosage of the pesticide will be reduced during application due to the good insecticidal activity of the amide compounds at low dosage, which is more conducive to environmental protection and has broad application prospect.

### FIELD OF THE INVENTION

This invention belongs to the field of insecticide, and relates to their production process and pesticidal utility.

### BACKGROUND OF THE INVENTION

The damage caused by pests is still very significant in agriculture and horticulture. The emergence of pests showing resistance to various insecticides and environmental impact of existing pesticides are both serious problems. Thus new insecticides with better insecticidal activity at low amount and environmental friendliness are continually needed to be developed.

The preparation and insecticidal activities of amide derivatives have been disclosed. CN105873901A disclosed the structures and insecticidal activities of KC1 and KC2 (i.e., compounds 128 and 2 of CN105873901A). CN110028423A disclosed the structure and insecticidal activities of KC3 (compound 5 of the patent). CN109497062A disclosed the structure and insecticidal activities of KC4 (compounds 62 in the patent). These disclosed compounds have insecticidal activities, but their insecticidal activities are not good or slow at low amount.

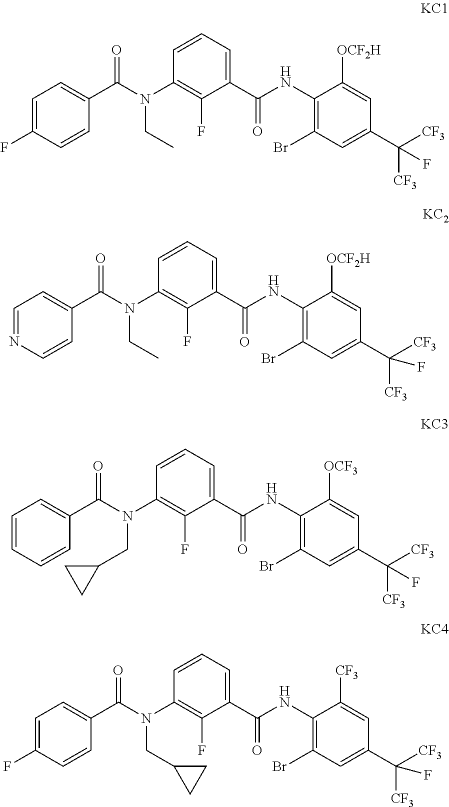

New insecticides with high insecticidal activities and quick efficacy at low amount are still needed to meet the demands of agriculture and forestry industry.

### SUMMARY OF THE INVENTION

In view of the shortcomings of the prior art, the object of this invention is to provide certain amide derivatives, their production process and pesticidal utility, namely, amide derivatives with difluoromethoxyl and/or pyridine moiety and their production process and pesticidal utility. The amide derivatives in this invention have good insecticidal activities at low amount and good quick-acting property. The amide derivatives in this invention are used at low amount, so they are more conducive to environmental protection.

In order to reach the above goals, this invention is specified by the following technical embodiments:

This invention provides amide compounds, which are defined by formula I:

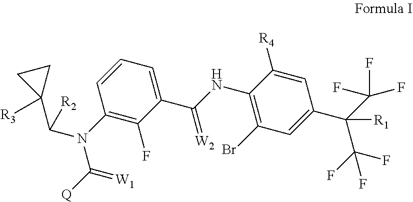

Wherein

Q is independently Q1, Q2, Q3 or Q4:

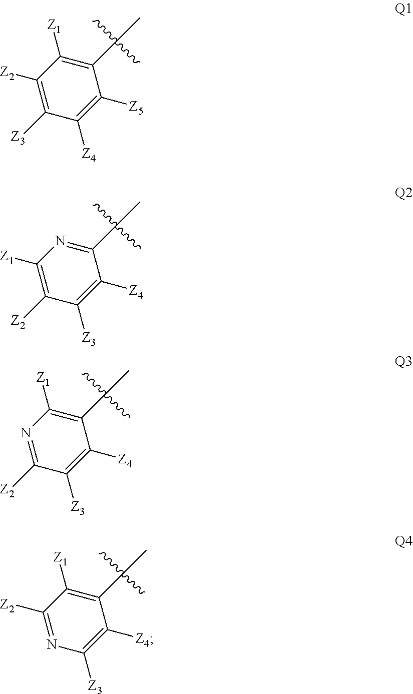

Z₁, Z₂, Z₃, Z₄, and Z₅ are independently of each other H, F, Cl, Br, I, CN, NO₂, C₁-C₆ alkyl, C₃-C₅ cycloalkyl, C₁-C₆ haloalkyl, C₃-C₈ halocycloalkyl, C₁-C₆ alkoxyl, C₁-C₆ haloalkoxyl, C₁-C₆ alkylsulfinyl, C₁-C₆ haloalkylsulfinyl, C₁-C₆ alkylsulfonyl or C₁-C₆ haloalkylsulfonyl;

### R₁ is H or F;

R₂ is H, C₁-C₆ alkyl, C₁-C₆ haloalkyl, C₃-C₈ cycloalkyl or C₃-C₈ halocycloalkyl;
R₃ is H or halogen;
R₄ is —OCF₂H or —CF₃, in a case when Q is Q1, R₄ is —OCF₂H;
W₁ and W₂ are independently of each other O or S.

Amide derivatives defined in formula I have excellent insecticidal activity and quick-acting property. Their insecticidal activity is good at low amount. Their insecticidal activity can be exerted after one day of application and the excellent insecticidal activity can be achieved at the third day after application. The good insecticidal activity at low amount of the amide derivatives in this invention can reduce the dose and the residue of pesticide, so they are more conducive to environmental protection.

Preference is given to compounds of formula I, in which,

Z₁, Z₂, Z₃, Z₄, and Z₅ are independently of each other H, F, Cl, Br, I, CN, NO₂, methyl, ethyl, n-propyl, i-propyl, c-propyl, n-butyl, t-butyl, i-butyl, n-pentyl, 1-methylbutyl, 2-methylbutyl, 3-methylbutyl, 1,1-dimethylpropyl, 1,2-dimethylpropyl, 2,2-dimethylpropyl, methoxyl, ethoxyl, n-propoxyl, i-propoxyl, t-butoxyl, trifluoromethyl, pentafluoroethyl, heptafluoropropyl, heptafluoroisopropyl, difluoromethoxyl, trifluoromethoxyl, pentafluoroethoxyl, methylsulfinyl, trifluoromethylsulfinyl, methylsulfonyl or trifluoromethylsulfonyl;
R₂ is H, methyl, ethyl, n-propyl, i-propyl, n-butyl, i-butyl, t-butyl, n-pentyl, 2-pentyl, neopentyl, isopentyl, 4-methyl-2-pentyl, n-hexyl, monofluoromethyl, difluoromethyl, trifluoromethyl, monochloromethyl, dichloromethyl, trichloromethyl, pentafluoroethyl, heptafluoroisopropyl, cyclopropyl, cyclobutyl, cyclopentyl, perfluorocyclopropyl, perfluoro cyclobutyl or perfluorocyclopentyl;

### R₃ is H, F or Cl.

The more preferred compounds of formula I, in which the amide compound is any one selected from table 1.

TABLE 1

| | | | | | | | | Appearance |
|---|---|---|---|---|---|---|---|---|
| Compound | | | | | | | | (melting |
| No. | Q | W₁ | W₂ | R₁ | R₂ | R₃ | R₄ | point: ° C.) |
| 1. | phenyl | O | O | F | H | H | OCF₂H | White solid |
| | | | | | | | | (155.1-156.6) |
| 2. | phenyl | O | O | F | Me | H | OCF₂H | |
| 3. | phenyl | O | O | F | CF₃ | H | OCF₂H | |
| 4. | phenyl | O | O | F | Me | Cl | OCF₂H | |
| 5. | phenyl | O | O | F | CF₃ | Cl | OCF₂H | |
| 6. | phenyl | O | O | F | CH₂Cl | H | OCF₂H | |
| 7. | phenyl | O | O | F | CH₂Cl | Cl | OCF₂H | |
| 8. | phenyl | O | O | F | CH₂F | H | OCF₂H | |
| 9. | phenyl | O | O | F | CH₂F | Cl | OCF₂H | |
| 10. | phenyl | S | O | F | H | H | OCF₂H | |
| 11. | phenyl | O | S | F | H | H | OCF₂H | |
| 12. | phenyl | S | S | F | H | H | OCF₂H | |
| 13. | phenyl | O | O | H | H | H | OCF₂H | White solid |
| | | | | | | | | (183.8-155.9) |
| 14. | 4-cyanophenyl | O | O | F | H | H | OCF₂H | Yellow oil |
| 15. | 4-cyanophenyl | O | O | F | Me | H | OCF₂H | |
| 16. | 4-cyanophenyl | O | O | F | CF₃ | H | OCF₂H | |
| 17. | 4-cyanophenyl | O | O | F | Me | Cl | OCF₂H | |
| 18. | 4-cyanophenyl | O | O | F | CF₃ | Cl | OCF₂H | |
| 19. | 4-cyanophenyl | O | O | F | CH₂Cl | H | OCF₂H | |
| 20. | 4-cyanophenyl | O | O | F | CH₂Cl | Cl | OCF₂H | |
| 21. | 4-cyanophenyl | O | O | F | CH₂F | H | OCF₂H | |
| 22. | 4-cyanophenyl | O | O | F | CH₂F | Cl | OCF₂H | |
| 23. | 4-cyanophenyl | S | O | F | H | H | OCF₂H | |
| 24. | 4-cyanophenyl | O | S | F | H | H | OCF₂H | |
| 25. | 4-cyanophenyl | S | S | F | H | H | OCF₂H | |
| 26. | 4-cyanophenyl | O | O | H | H | H | OCF₂H | White solid |
| | | | | | | | | (133.1-135.5) |
| 27. | 4-(trifluoromethyl)phenyl | O | O | F | H | H | OCF₂H | White solid |
| | | | | | | | | (104.9-107.1) |
| 28. | 4-(trifluoromethyl)phenyl | O | O | H | H | H | OCF₂H | Colorless oil |
| 29. | 4-(pentafluoroethyl)phenyl | O | O | F | H | H | OCF₂H | |
| 30. | 4-(heptafluoroisopropyl)phenyl | O | O | F | H | H | OCF₂H | |
| 31. | 4-fluorophenyl | O | O | F | H | H | OCF₂H | Yellow solid |
| | | | | | | | | (94.0-96.0) |
| 32. | 4-fluorophenyl | O | O | F | Me | H | OCF₂H | |
| 33. | 4-fluorophenyl | O | O | F | CF₃ | H | OCF₂H | |
| 34. | 4-fluorophenyl | O | O | F | Me | Cl | OCF₂H | |
| 35. | 4-fluorophenyl | O | O | F | CF₃ | Cl | OCF₂H | |
| 36. | 4-fluorophenyl | O | O | F | CH₂Cl | H | OCF₂H | |
| 37. | 4-fluorophenyl | O | O | F | CH₂Cl | Cl | OCF₂H | |
| 38. | 4-fluorophenyl | O | O | F | CH₂F | H | OCF₂H | |
| 39. | 4-fluorophenyl | O | O | F | CH₂F | Cl | OCF₂H | |
| 40. | 4-fluorophenyl | S | O | F | H | H | OCF₂H | |
| 41. | 4-fluorophenyl | O | S | F | H | H | OCF₂H | |
| 42. | 4-fluorophenyl | S | S | F | H | H | OCF₂H | |
| 43. | 4-fluorophenyl | O | O | H | H | H | OCF₂H | White solid |
| | | | | | | | | (135.8-137.6) |
| 44. | 4-chlorophenyl | O | O | F | H | H | OCF₂H | White solid |
| | | | | | | | | (137.8-139.7) |
| 45. | 4-chlorophenyl | O | O | H | H | H | OCF₂H | |
| 46. | 4-bromophenyl | O | O | F | H | H | OCF₂H | |
| 47. | 4-bromophenyl | O | O | H | H | H | OCF₂H | |
| 48. | 4-iodophenyl | O | O | F | H | H | OCF₂H | |
| 49. | 4-iodophenyl | O | O | H | H | H | OCF₂H | |
| 50. | 4-(methyl)phenyl | O | O | F | H | H | OCF₂H | |
| 51. | 4-(methyl)phenyl | O | O | H | H | H | OCF₂H | |
| 52. | 4-(isopropyl)phenyl | O | O | F | H | H | OCF₂H | |
| 53. | 4-(isopropyl)phenyl | O | O | H | H | H | OCF₂H | |
| 54. | 4-(cyclopropyl)phenyl | O | O | F | H | H | OCF₂H | |
| 55. | 4-(t-butyl)phenyl | O | O | F | H | H | OCF₂H | White solid |
| | | | | | | | | (180.0-181.0) |
| 56. | 4-(t-butyl)phenyl | O | O | H | H | H | OCF₂H | |
| 57. | 4-(methoxyl)phenyl | O | O | F | H | H | OCF₂H | |
| 58. | 4-(methoxyl)phenyl | O | O | H | H | H | OCF₂H | |
| 59. | 4-(isopropoxyl)phenyl | O | O | F | H | H | OCF₂H | |
| 60. | 4-(isopropoxyl)phenyl | O | O | H | H | H | OCF₂H | |
| 61. | 4-(methylsulfonyl)phenyl | O | O | F | H | H | OCF₂H | |
| 62. | 4-(methylsulfonyl)phenyl | O | O | H | H | H | OCF₂H | Yellow solid |
| | | | | | | | | (187-188) |
| 63. | 4-(trifluoromethylsulfonyl)phenyl | O | O | F | H | H | OCF₂H | |
| 64. | 4-(trifluoromethylsulfonyl)phenyl | O | O | H | H | H | OCF₂H | |
| 65. | 4-(trifluoromethoxyl)phenyl | O | O | F | H | H | OCF₂H | |
| 66. | 4-(trifluoromethoxyl)phenyl | O | O | H | H | H | OCF₂H | |
| 67. | 4-(difluoromethoxyl)phenyl | O | O | F | H | H | OCF₂H | |
| 68. | 4-(difluoromethoxyl)phenyl | O | O | H | H | H | OCF₂H | |
| 69. | 4-(pentafluorothoxyl)phenyl | O | O | F | H | H | OCF₂H | |
| 70. | 4-(pentafluorothoxyl)phenyl | O | O | H | H | H | OCF₂H | |
| 71. | 2-fluorophenyl | O | O | F | H | H | OCF₂H | |
| 72. | 2-fluorophenyl | O | O | H | H | H | OCF₂H | |
| 73. | 3-fluorophenyl | O | O | F | H | H | OCF₂H | |
| 74. | 3-fluorophenyl | O | O | H | H | H | OCF₂H | |
| 75. | 2,3-difluorophenyl | O | O | F | H | H | OCF₂H | |
| 76. | 2,3-difluorophenyl | O | O | H | H | H | OCF₂H | |
| 77. | 2,4-difluorophenyl | O | O | F | H | H | OCF₂H | Yellow solid |
| | | | | | | | | (85.5-87.2) |
| 78. | 2,4-difluorophenyl | O | O | F | H | H | OCF₂H | White solid |
| | | | | | | | | (129.3-131.3) |
| 79. | 2,5-difluorophenyl | O | O | F | H | H | OCF₂H | |
| 80. | 2,5-difluorophenyl | O | O | H | H | H | OCF₂H | |
| 81. | 2,6-difluorophenyl | O | O | F | H | H | OCF₂H | White solid |
| | | | | | | | | (144.8-146.3) |
| 82. | 2,6-difluorophenyl | O | O | H | H | H | OCF₂H | |
| 83. | 3,4-difluorophenyl | O | O | F | H | H | OCF₂H | White solid |
| | | | | | | | | (148.5-151.1) |
| 84. | 3,4-difluorophenyl | O | O | H | H | H | OCF₂H | |
| 85. | 3,5-difluorophenyl | O | O | F | H | H | OCF₂H | Yellow solid |
| | | | | | | | | (71.0-73.0) |
| 86. | 3,5-difluorophenyl | O | O | H | H | H | OCF₂H | |
| 87. | 2,4,6-trifluorophenyl | O | O | F | H | H | OCF₂H | |
| 88. | 2,4,6-trifluorophenyl | O | O | H | H | H | OCF₂H | |
| 89. | 2,3,4-trifluorophenyl | O | O | F | H | H | OCF₂H | |
| 90. | 2,4,5-trifluorophenyl | O | O | F | H | H | OCF₂H | |
| 91. | 2,3,5-trifluorophenyl | O | O | F | H | H | OCF₂H | |
| 92. | 2,3,5-trifluorophenyl | O | O | H | H | H | OCF₂H | |
| 93. | 2,3,6-trifluorophenyl | O | O | F | H | H | OCF₂H | |
| 94. | 2,3,6-trifluorophenyl | O | O | H | H | H | OCF₂H | |
| 95. | 4-nitrophenyl | O | O | F | H | H | OCF₂H | |
| 96. | 4-nitrophenyl | O | O | H | H | H | OCF₂H | |
| 97. | phenyl | O | O | F | F | H | OCF₂H | |
| 98. | 2-cyanophenyl | O | O | F | H | H | OCF₂H | Yellow solid |
| | | | | | | | | (125.8-127.4) |
| 99. | pyridin-2-yl | O | O | F | H | H | CF₃ | |
| 100. | pyridin-3-yl | O | O | F | H | H | CF₃ | White solide |
| 101. | pyridin-4-yl | O | O | F | H | H | CF₃ | White solide |
| 102. | 2-chloropyridin-3-yl | O | O | F | H | H | CF₃ | White solide |
| 103. | 2-fluoropyridin-3-yl | O | O | F | H | H | CF₃ | |
| 104. | 2-methylpyridin-3-yl | O | O | F | H | H | CF₃ | |
| 105. | 6-chloropyridin-3-yl | O | O | F | H | H | CF₃ | Yellow solid |
| 106. | 6-fluoropyridin-3-yl | O | O | F | H | H | CF₃ | Yellow solid |
| 107. | 6-methylpyridin-3-yl | O | O | F | H | H | CF₃ | |
| 108. | 2-chloro-6-trifluoromethylpyridin-3-y1 | O | O | F | H | H | CF₃ | Yellow liquid |
| 109. | 2-chloro-6-methylpyridin-3-y1 | O | O | F | H | H | CF₃ | White solide |
| 110. | 2-chloropyridin-4-yl | O | O | F | H | H | CF₃ | Yellow liquid |
| 111. | 2-fluoropyridin-4-yl | O | O | F | H | H | CF₃ | Light yellow |
| | | | | | | | | solid |
| 112. | pyridin-3-yl | O | O | F | H | H | OCF₂H | Yellow solid |
| 113. | pyridin-4-yl | O | O | F | H | H | OCF₂H | Yellow solid |
| 114. | pyridin-2-yl | O | O | F | H | H | OCF₂H | |
| 115. | 2-chloropyridin-3-yl | O | O | F | H | H | OCF₂H | |
| 116. | 2-fluoropyridin-3-yl | O | O | F | H | H | OCF₂H | |
| 117. | 6-chloropyridin-3-yl | O | O | F | H | H | OCF₂H | Yellow oil |
| 118. | 6-fluoropyridin-3-yl | O | O | F | H | H | OCF₂H | Yellow oil |
| 119. | 2-chloropyridin-4-yl | O | O | F | H | H | OCF₂H | Yellow solid |
| 120. | 2-fluoropyridin-4-yl | O | O | F | H | H | OCF₂H | White solid |
| | | | | | | | | (104.0-105.8) |
| 121. | pyridin-3-yl | O | O | F | Me | H | CF₃ | White solid |
| 122. | pyridin-4-yl | O | O | F | Me | H | CF₃ | Yellow solid |
| 123. | 6-fluoropyridin-3-yl | O | O | F | Me | H | CF₃ | Yellow solid |
| 124. | 2-chloropyridin-4-yl | O | O | F | Me | H | CF₃ | Yellow solid |
| 125. | 2-fluoropyridin-4-yl | O | O | F | Me | H | CF₃ | |
| 126. | pyridin-2-yl | O | O | F | c-Pr | H | CF₃ | |
| 127. | pyridin-3-yl | O | O | F | c-Pr | H | CF₃ | |
| 128. | pyridin-4-yl | O | O | F | c-Pr | H | CF₃ | |
| 129. | 6-fluoropyridin-3-yl | O | O | F | c-Pr | H | CF₃ | |
| 130. | 2-chloropyridin-4-yl | O | O | F | c-Pr | H | CF₃ | |
| 131. | 2-fluoropyridin-4-yl | O | O | F | c-Pr | H | CF₃ | |
| 132. | 6-fluoropyridin-3-yl | O | O | F | 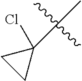 [SMILES: *C1(Cl)CC1] | H | CF₃ | |
| 133. | 2-chloropyridin-4-yl | O | O | F | 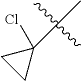 [SMILES: *C1(Cl)CC1] | H | CF₃ | |
| 134. | 2-fluoropyridin-4-yl | O | O | F | 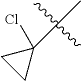 [SMILES: *C1(Cl)CC1] | H | CF₃ | |
| 135. | 6-fluoropyridin-3-yl | O | O | F | 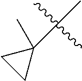 [SMILES: *C1(C)CC1] | H | CF₃ | |
| 136. | 2-chloropyridin-4-yl | O | O | F | 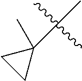 [SMILES: *C1(C)CC1] | H | CF₃ | |
| 137. | 2-fluoropyridin-4-yl | O | O | F | 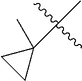 [SMILES: *C1(C)CC1] | H | CF₃ | |
| 138. | 2-chloropyridin-4-yl | O | O | F | Me | H | OCF₂H | |
| 139. | 2-fluoropyridin-4-yl | O | O | F | Me | H | OCF₂H | |
| 140. | 6-fluoropyridin-3-yl | O | O | F | Me | H | OCF₂H | |
| 141. | 2-chloropyridin-4-yl | O | O | F | c-Pr | H | OCF₂H | |
| 142. | 2-fluoropyridin-4-yl | O | O | F | c-Pr | H | OCF₂H | |
| 143. | 6-fluoropyridin-3-yl | O | O | F | c-Pr | H | OCF₂H | |
| 144. | 5-trifluoromethylpyridin-2-yl | O | O | F | H | H | OCF₂H | Yellow solid |
| | | | | | | | | (146.2-147.3) |
| 145. | 5-fluoropyridin-2-yl | O | O | F | H | H | OCF₂H | Yellow oil |
| 146. | 5-chloropyridin-2-yl | O | O | F | H | H | OCF₂H | Yellow oil |
| 147. | 5-bromopyridin-2-yl | O | O | F | H | H | OCF₂H | Brown oil |
| 148. | 5-nitropyridin-2-yl | O | O | F | H | H | OCF₂H | Yellow oil |
| 149. | 5-cyanopyridin-2-yl | O | O | F | H | H | OCF₂H | Yellow oil |
| 150. | 5-trifluoromethylpyridin-2-yl | O | O | F | H | H | CF₃ | Yellow solid |
| | | | | | | | | (147.2-148.8) |
| 151. | 5-fluoropyridin-3-yl | O | O | F | H | H | CF₃ | |
| 152. | 5-chloropyridin-2-yl | O | O | F | H | H | CF₃ | Yellow solid |
| | | | | | | | | (61.4-63.1) |
| 153. | 5-bromopyridin-2-yl | O | O | F | H | H | CF₃ | Yellow solid |
| | | | | | | | | (134.0-135.6) |
| 154. | 5-nitropyridin-2-yl | O | O | F | H | H | CF₃ | Yellow oil |
| 155. | 5-cyanopyridin-2-yl | O | O | F | H | H | CF₃ | |
| 156. | pyridin-3-yl | O | O | H | H | H | CF₃ | White solid |
| | | | | | | | | (162.3-164.5) |
| 157. | pyridin-4-yl | O | O | H | H | H | CF₃ | White solid |
| | | | | | | | | (189.1-191.5) |
| 158. | 2-chloropyridin-3-yl | O | O | H | H | H | CF₃ | |
| 159. | 2-fluoropyridin-3-yl | O | O | H | H | H | CF₃ | |
| 160. | 6-chloropyridin-3-yl | O | O | H | H | H | CF₃ | |
| 161. | 6-fluoropyridin-3-yl | O | O | H | H | H | CF₃ | Yellow solid |
| | | | | | | | | (144.1-145.9) |
| 162. | 2-chloropyridin-4-yl | O | O | H | H | H | CF₃ | |
| 163. | 2-fluoropyridin-4-yl | O | O | H | H | H | CF₃ | |
| 164. | pyridin-3-yl | O | O | H | H | H | OCF₂H | White solid |
| | | | | | | | | (140.1-142.0) |
| 165. | pyridin-4-yl | O | O | H | H | H | OCF₂H | Yellow oil |
| 166. | 2-trifluoromethylpyridin-3-yl | O | O | H | H | H | OCF₂H | |
| 167. | 2-fluoropyridin-3-yl | O | O | H | H | H | OCF₂H | |
| 168. | 6-chloropyridin-3-yl | O | O | H | H | H | OCF₂H | Yellow oil |
| 169. | 6-fluoropyridin-3-yl | O | O | H | H | H | OCF₂H | White solid |
| | | | | | | | | (66.7-67.8) |
| 170. | 2-chloropyridin-4-yl | O | O | H | H | H | OCF₂H | Yellow liquid |
| 171. | 2-fluoropyridin-4-yl | O | O | H | H | H | OCF₂H | |
| 172. | 2-trifluoromethylpyridin-4-yl | O | O | H | H | H | OCF₂H | |
| 173. | 3-chloropyridin-2-yl | O | O | F | H | H | OCF₂H | White solid |
| | | | | | | | | (132.1-133.2) |
| 174. | 3-chloropyridin-2-yl | O | O | F | H | H | CF₃ | Yellow solid |
| | | | | | | | | (80.0-81.5) |
| 175. | 3,5-dichloropyridin-2-yl | O | O | F | H | H | CF₃ | Yellow oil |
| 176. | 3,5-dichloropyridin-2-yl | O | O | F | H | H | OCF₂H | Yellow oil |
| 177. | 5-(methylsulfonyl)pyridin-2-y1 | O | O | F | H | H | OCF₂H | Yellow oil |
| 178. | 6-fluoropyridin-2-y1 | O | O | F | H | H | OCF₂H | Brown solid |
| | | | | | | | | (138.0-139.0) |
| 179. | 6-fluoropyridin-2-yl | O | O | F | H | H | CF₃ | Yellow oil |
| 180. | 4-cyano-3-methylphenyl | O | O | F | H | H | OCF₂H | Yellow oil |
| 181. | 4-cyano-2-fluorophenyl | O | O | F | H | H | OCF₂H | Yellow oil |
| 182. | 4-cyano-2-methylphenyl | O | O | F | H | H | OCF₂H | Yellow solid |
| | | | | | | | | (99.8-100.7) |

Notes:
‘H’ represents hydrogen atom, ‘O’ represents oxygen atom, ‘S’ represents sulfur atom,
‘F’ represents fluorine atom, ‘Cl’ represents chlorine atom, ‘Br’ represents bromine atom,
‘Me’ represents methyl, ‘CH2Cl’ represents monochloromethyl, ‘CH2F’ represents
monofluoromethyl, ‘CF3’ represents trifluoromethyl, ‘OCF2H’ represents difluoromethoxyl.

The further more preferred compounds of formula I, in which,

Z₁, Z₂, Z₃, Z₄, and Z₅ are independently of each other H, F, Cl, Br, I, CN, NO₂, methyl, trifluoromethyl, difluoromethoxyl, trifluoromethoxyl, methylsulfonyl or trifluoromethyl sulfonyl;

### R₁ is H or F;

R₂ is H or methyl;

### R₃ is H or Cl;

W₁ and W₂ are independently of each other O.

The particular preferred compounds of formula I are selected from any compound below:

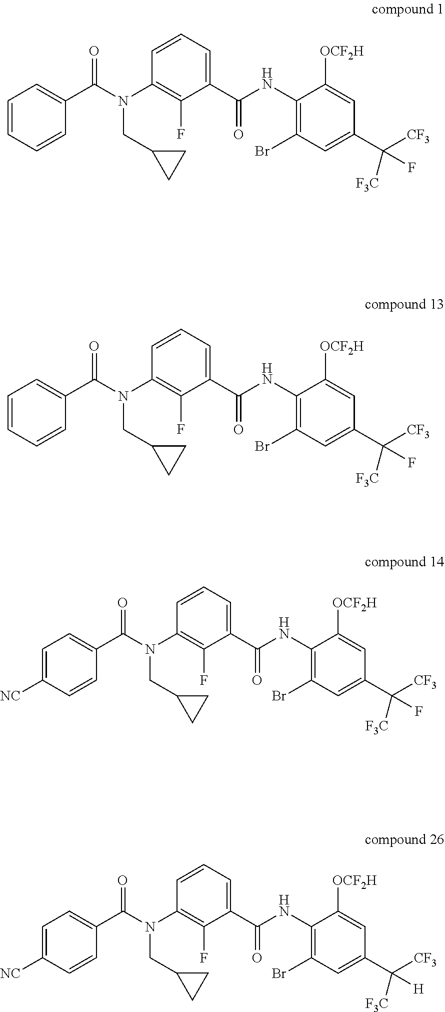

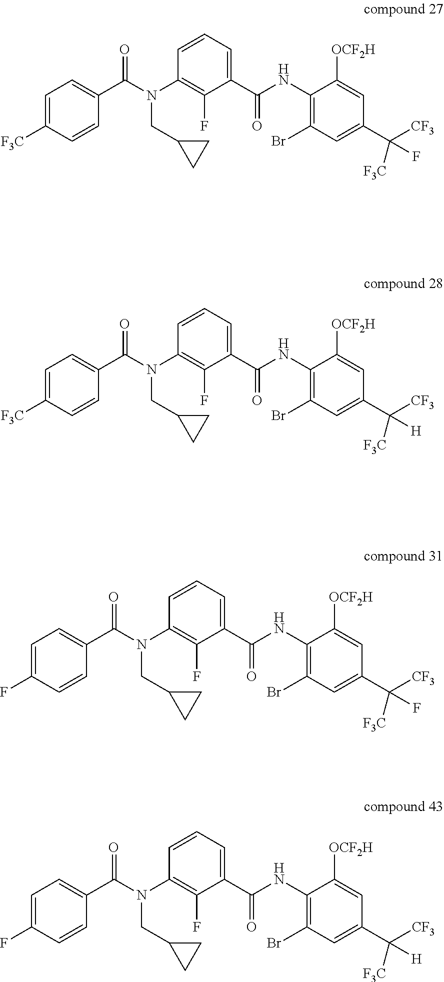

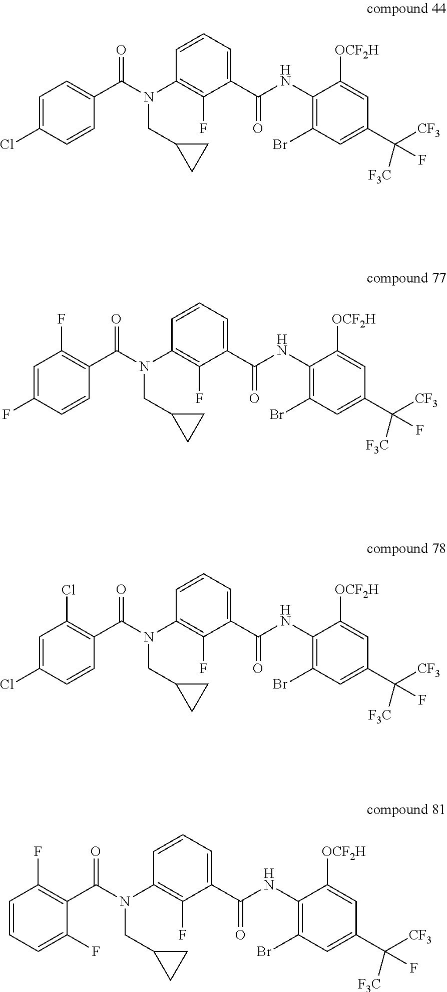

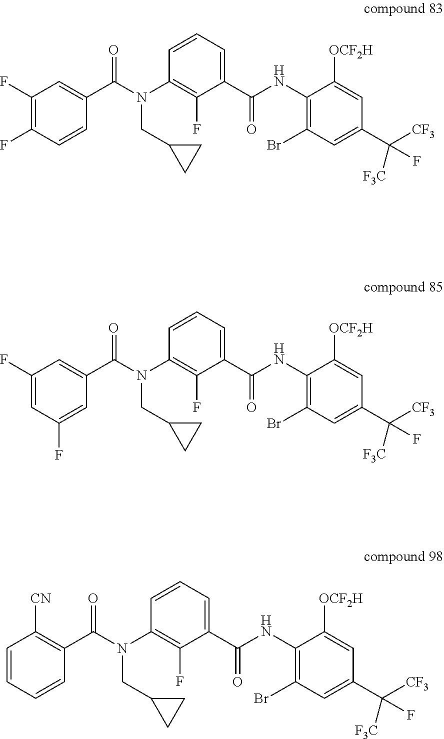

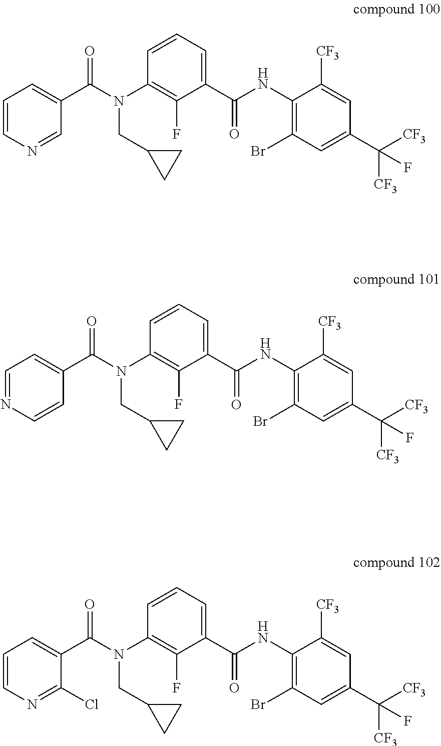 [SMILES: O=C(Nc1c(Br)cc(C(F)(C(F)(F)F)C(F)(F)F)cc1C(F)(F)F)c1cccc(N(CC2CC2)C(=O)c2cccnc2)c1F ; O=C(Nc1c(Br)cc(C(F)(C(F)(F)F)C(F)(F)F)cc1C(F)(F)F)c1cccc(N(CC2CC2)C(=O)c2ccncc2)c1F ; O=C(Nc1c(Br)cc(C(F)(C(F)(F)F)C(F)(F)F)cc1C(F)(F)F)c1cccc(N(CC2CC2)C(=O)c2cccnc2Cl)c1F]

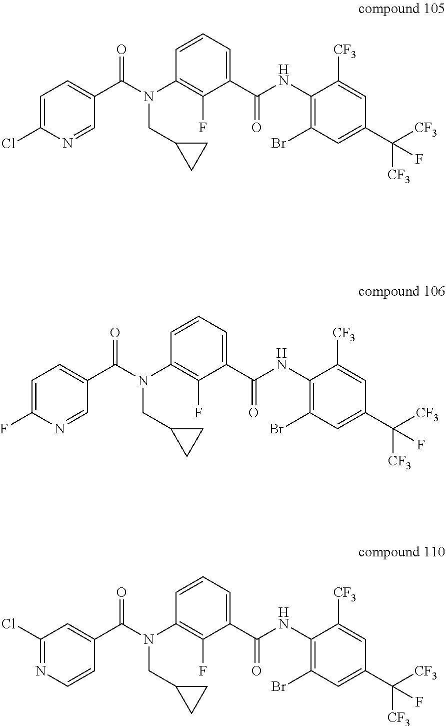 [SMILES: O=C(Nc1c(Br)cc(C(F)(C(F)(F)F)C(F)(F)F)cc1C(F)(F)F)c1cccc(N(CC2CC2)C(=O)c2ccc(Cl)nc2)c1F ; O=C(Nc1c(Br)cc(C(F)(C(F)(F)F)C(F)(F)F)cc1C(F)(F)F)c1cccc(N(CC2CC2)C(=O)c2ccc(F)nc2)c1F ; O=C(Nc1c(Br)cc(C(F)(C(F)(F)F)C(F)(F)F)cc1C(F)(F)F)c1cccc(N(CC2CC2)C(=O)c2ccnc(Cl)c2)c1F]

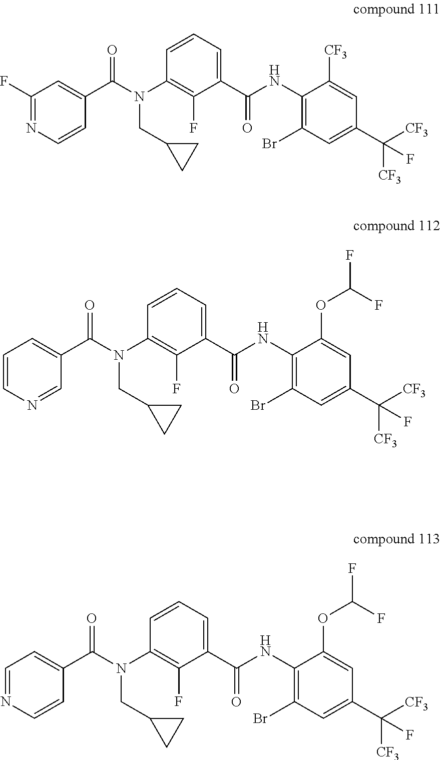 [SMILES: O=C(Nc1c(Br)cc(C(F)(C(F)(F)F)C(F)(F)F)cc1C(F)(F)F)c1cccc(N(CC2CC2)C(=O)c2ccnc(F)c2)c1F ; O=C(Nc1c(Br)cc(C(F)(C(F)(F)F)C(F)(F)F)cc1OC(F)F)c1cccc(N(CC2CC2)C(=O)c2cccnc2)c1F ; O=C(Nc1c(Br)cc(C(F)(C(F)(F)F)C(F)(F)F)cc1OC(F)F)c1cccc(N(CC2CC2)C(=O)c2ccncc2)c1F]

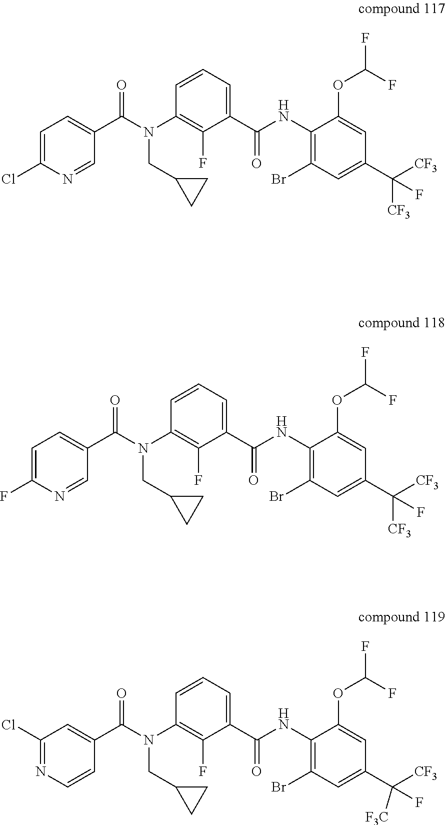 [SMILES: O=C(Nc1c(Br)cc(C(F)(C(F)(F)F)C(F)(F)F)cc1OC(F)F)c1cccc(N(CC2CC2)C(=O)c2ccc(Cl)nc2)c1F ; O=C(Nc1c(Br)cc(C(F)(C(F)(F)F)C(F)(F)F)cc1OC(F)F)c1cccc(N(CC2CC2)C(=O)c2ccc(F)nc2)c1F ; O=C(Nc1c(Br)cc(C(F)(C(F)(F)F)C(F)(F)F)cc1OC(F)F)c1cccc(N(CC2CC2)C(=O)c2ccnc(Cl)c2)c1F]

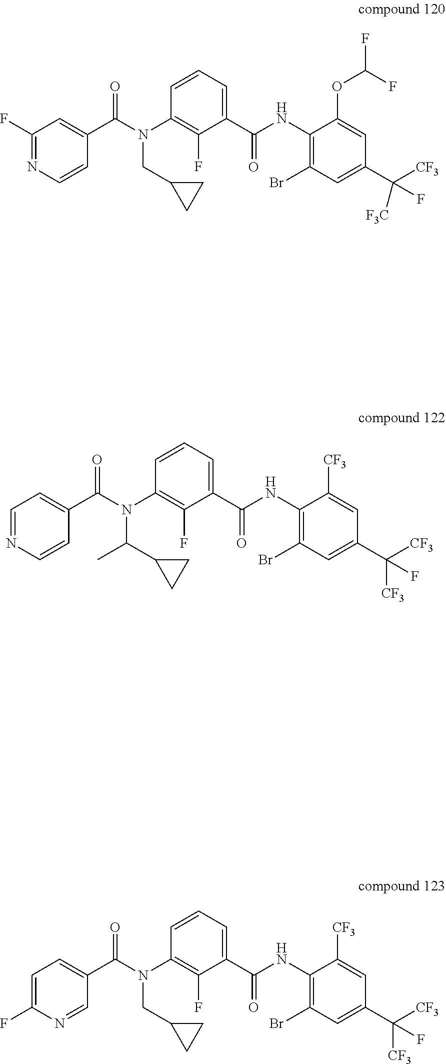 [SMILES: O=C(Nc1c(Br)cc(C(F)(C(F)(F)F)C(F)(F)F)cc1OC(F)F)c1cccc(N(CC2CC2)C(=O)c2ccnc(F)c2)c1F ; CC(C1CC1)N(C(=O)c1ccncc1)c1cccc(C(=O)Nc2c(Br)cc(C(F)(C(F)(F)F)C(F)(F)F)cc2C(F)(F)F)c1F ; O=C(Nc1c(Br)cc(C(F)(C(F)(F)F)C(F)(F)F)cc1C(F)(F)F)c1cccc(N(CC2CC2)C(=O)c2ccc(F)nc2)c1F]

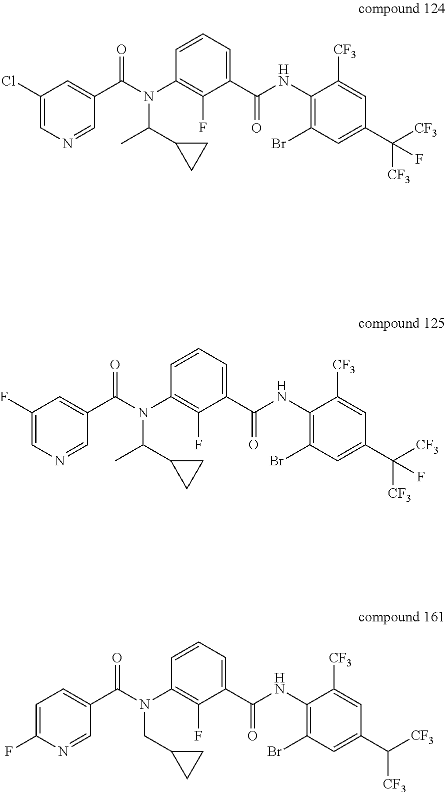 [SMILES: CC(C1CC1)N(C(=O)c1cncc(Cl)c1)c1cccc(C(=O)Nc2c(Br)cc(C(F)(C(F)(F)F)C(F)(F)F)cc2C(F)(F)F)c1F ; CC(C1CC1)N(C(=O)c1cncc(F)c1)c1cccc(C(=O)Nc2c(Br)cc(C(F)(C(F)(F)F)C(F)(F)F)cc2C(F)(F)F)c1F ; O=C(Nc1c(Br)cc(C(C(F)(F)F)C(F)(F)F)cc1C(F)(F)F)c1cccc(N(CC2CC2)C(=O)c2ccc(F)nc2)c1F]

In which the numbers of the above compounds are corresponding to the numbers in the table 1. The alkyl in present invention represents a straight-chain or branched alkyl group, for example methyl, ethyl, n-propyl, i-propyl, n-butyl, i-butyl, s-butyl, t-butyl, n-pentyl, i-pentyl, n-hexyl, and the like. Haloalkyl represents alkyl substituted by one or more halogen atoms which may be the same as or different from each other. Alkoxyl represents the alkyl substituted by oxygen atom, for example, methoxyl, ethoxyl, n-propoxyl, i-propoxyl, t-butxoyl, and the like. Haloalkoxyl represents alkoxyl substituted by one or more halogen atoms which may be the same as or different from each other. Halogen refers to F, Cl, Br or I.

As used herein, the term “C₁-C₆ alkyl” represents straight-chain or branched alkyl group having 1 to 6 carbon atoms, including but not limiting to methyl, ethyl, n-propyl, i-propyl, n-butyl, i-butyl, s-butyl, t-butyl, n-pentyl, i-pentyl, n-hexyl and the like. The term “C₁-C₆ alkoxyl” represents straight-chain or branched alkoxyl group having 1 to 6 carbon atoms, including but not limiting to methoxyl, ethoxyl, n-propoxyl, t-butxoyl, and the like. “C₁-C₆ haloalkyl” represents a straight-chain or branched alkyl group having 1 to 6 carbon atoms, that is substituted with one or more halogen atoms which may be the same as or different from each other, including but not limiting to trifluoromethyl, pentafluoroethyl, heptafluoropropyl, heptafluoroisopropyl and the like. The term “C₃-C₈ cycloalkyl” represents cycloalkyl group having 3 to 8 carbon atoms, including but not limiting to cyclopropyl, cyclobutyl, cyclopentyl, cyclohexyl, cycloheptanyl, cyclooctyl and the like. “C₃-C₈ halocycloalkyl” represents cycloalkyl group having 3 to 8 carbon atoms, which is substituted with one or more halogen atoms which may be the same as or different from each other, including but not limiting to 1-chlorocyclopropyl, 1-fluorocyclopropyl, perfluorocyclopropyl, 1-chlorocyclopentyl, 1-chlorocyclobutyl and the like.

C₁-C₆, C₃-C₈ and the like in front of specific group refer to the number of carbon atoms contained in the group, for example, C₁-C₆ represents the group containing 1, 2, 3, 4, 5 or 6 carbon atoms, C₃-C₈ represents the group containing 3, 4, 5, 6, 7 or 8 carbon atoms, and the like.

Furthermore, “Me” represents methyl, “c-Pr” represents cyclopropyl, “CF₃” represents trifluoromethyl, “OCF₃” represents trifluoromethoxyl, “OCF₂H” represents difluoromethoxyl, “H” represents hydrogen atom, “F” represents fluorine atom, “Cl” represents chlorine atom, “Br” represents bromine atom, “I” represents iodine atom, “O” represents oxygen atom, “S” represents sulfur atom, “OMe” represents methoxyl, “CN” represents cyano, “NO₂” represents nitro.

Compounds of formula I can be prepared by following methods. Definitions of each group in the reactions are the same as the above, unless otherwise specified.

### Preparation Method 1

The structures of general formula I according to this invention are as following, which can be prepared by the following methods.

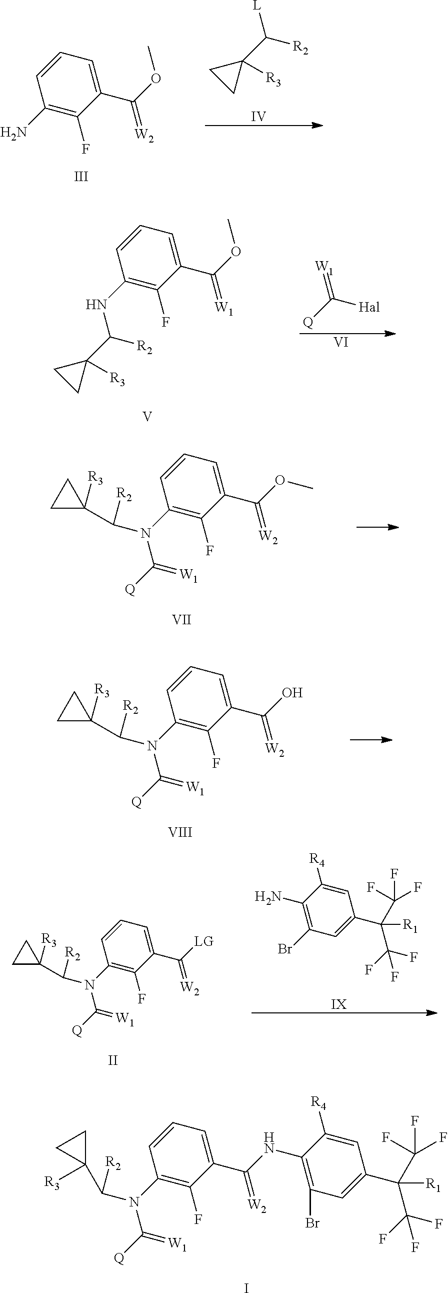

Wherein, the LG is selected from the group consisting of F, Cl, Br, C₁-C₁₂ alkoxyl, C₁-C₁₂ alkoxyl acyloxyl or C₁-C₁₂ alkyl acyloxyl; Hal is selected from the group consisting of F, Cl, Br or I; L is selected from Cl, Br, I or C₁-C₆ alkyl sulfonate group; R₁, R₂, R₃, R₄, Q, W₁, W₂ are defined identically as above.

Formula III+Formula IV→Formula V 1-(i):

Preferably, the compound represented by Formula III can be suitably selected in the range of 0.5 to 2 molar equivalents based on the compound represented by Formula IV.

In the process of the reaction 1-(i), a base can be used, including organic bases and/or inorganic bases.

Preferably, examples of the organic bases include any one of triethylamine, N, N-diisopropylethylamine, pyridine, sodium methoxide, sodium ethoxide or a combination of at least two thereof.

Preferably, examples of the inorganic bases include any one of sodium carbonate, potassium carbonate, sodium hydroxide, potassium hydroxide or sodium hydride or a combination of at least two thereof.

Preferably, solvents of the reaction 1-(i) include any one of dichloromethane, toluene, ethyl acetate, acetone, tetrahydrofuran, N, N-dimethylformamide, dimethyl sulfoxide or a combination of at least two thereof.

Preferably, the reaction temperature of the reaction 1-(i) can be appropriately selected within the range from room temperature to the boiling point of the solvent used, such as 25° C., 50° C., 75° C., 90° C. or the boiling point, i.e., the reflux temperature of the solvent used.

Preferably, the reaction time of 1-(i) can be appropriately selected within the range from half an hour to 48 hours.

Formula V+Formula VI→Formula VII 1-(ii):

By reacting a compound represented by the general Formula V with a compound represented by the general Formula VI, a compound represented by the general Formula VII can be prepared.

Preferably, the compound represented by Formula V can be suitably selected in the range of 0.5 to 2 molar equivalents based on the compound represented by Formula VI.

In the process of the reaction 1-(ii), a base can be used, including organic bases and or inorganic bases.

Preferably, examples of the organic bases include any one of triethylamine, N, N-diisopropylethylamine, pyridine, sodium methoxide, sodium ethoxide, or a combination of at least two thereof.

Preferably, examples of the inorganic bases include any one of sodium carbonate, potassium carbonate, sodium hydroxide, potassium hydroxide or sodium hydride or a combination of at least two thereof.

Preferably, solvents of the reaction 1-(ii) include any one of dichloromethane, chloroform, toluene, ethyl acetate, tetrahydrofuran, N, N-dimethylformamide, dimethyl sulfoxide or a combination of at least two thereof.

Preferably, the reaction temperature of t 1-(ii) can be appropriately selected within the range from −10° C. to the boiling point of the solvent used, such as −10° C., 0° C., 10° C., 30° C., 50° C., 75° C., 90° C. or boiling point, i.e., the reflux temperature of the solvent used.

Preferably, the reaction time of 1-(ii) can be appropriately selected within the range from half an hour to 48 hours.

Formula VII→Formula VIII 1-(iii):

By hydrolysing a compound represented by the general Formula VII, a compound represented by the general Formula VIII can be obtained.

The hydrolysis reaction of 1-(iii) is conducted in any one of water, methanol, ethanol, tetrahydrofuran, dioxane or the mixture of at least two thereof.

Preferably, in the process of the reaction 1-(iii), a base can also be used, preferably including lithium hydroxide, sodium hydroxide or potassium hydroxide.

Preferably, the base can be suitably selected in the range of 1 to 5 molar equivalents based on the compound represented by Formula VII.

Formula VIII→Formula II 1-(iv):

A compound represented by the general formula II having a leaving group can be prepared by a well-known method reacting a compound represented by the general formula VIII with thionyl chloride, oxalyl chloride, triphosgene or the like.

Formula II+Formula IX→Formula I 1-(v):

By reacting a compound represented by the general Formula II with a compound represented by the general Formula IX, a compound represented by the general Formula I can be prepared.

Preferably, the compound represented by Formula II can be suitably selected in the range of 0.5 to 2 molar equivalents based on the compound represented by Formula IX.

In the process of the reaction 1-(v), a base can be used, including organic bases and/or inorganic bases.

Preferably, examples of the organic bases include any one of triethylamine, N,N-diisopropylethylamine, pyridine, piperidine, 4-N, N-dimethylaminopyridine, alkali alcoholate, lithium amino or a combination of at least two thereof.

Preferably, the alkali alcoholate is sodium methoxide and/or sodium ethoxide. And the lithium amino is lithium diisopropylamide.

Preferably, the inorganic bases include any one of alkali metal hydroxides, carbonates, phosphates or a combination of at least two thereof.

Preferably, the alkali metal hydroxides contain any one of lithium hydroxide, sodium hydroxide, potassium hydroxide or a combination of at least two thereof. Preferably, the alkali metal carbonates include any one of sodium bicarbonate, sodium carbonate, potassium carbonate or a combination of at least two thereof. Preferably, the alkali metal phosphates include dipotassium hydrogen phosphate and/or trisodium phosphate.

Preferably, the solvents of 1-(v) may be any of those which do not inhibit the present reaction significantly. The solvent can include any one of halogenated hydrocarbons, aromatic hydrocarbons, chained or cyclic ethers, esters, ketones, nitriles, polar aprotic inert solvents or a combination of at least two thereof.

Preferably, the halogenated hydrocarbons include any one of methylene dichloride, chloroform or carbon tetrachloride or a combination of at least two thereof. Preferably, the aromatic hydrocarbons include any one of benzene, toluene, xylene, chlorobenzene or dichlorobenzene or a combination of at least two thereof. Preferably, the chained or cyclic ethers include any one of ether, tetrahydrofuran, dioxane or 1,2-dimethoxyethane or a combination of at least two thereof. Preferably, the esters include ethyl acetate and/or butyl acetate. Preferably, the ketones include any one of acetone, methyl isobutyl ketone, cyclohexanone or a combination of at least two thereof. Preferably, the nitriles include acetonitrile and/or acrylonitrile. Preferably, the polar aprotic inert solvents include any one of 1, 3-dimethyl-2-imidazolinone, sulfolane, dimethyl sulfoxide, N, N-dimethylformamide, N-methylpyrrolidone, N,N-dimethylacetamide or hexamethylphosphamide or a combination of at least two thereof.

Preferably, the reaction temperature of the reaction 1-(v) can be appropriately selected within the range from −70° C. to the boiling point of the solvent used, such as −70° C., −50° C., −10° C., 0° C., 45° C., 90° C. or the boiling point, i.e., the reflux temperature of the solvent used.

Preferably, the reaction time of the reaction 1-(v) can be appropriately selected within the range from half an hour to 48 hours.

### Preparation Method 2

The compounds of general formula I of this invention can be prepared by an alternative method shown below:

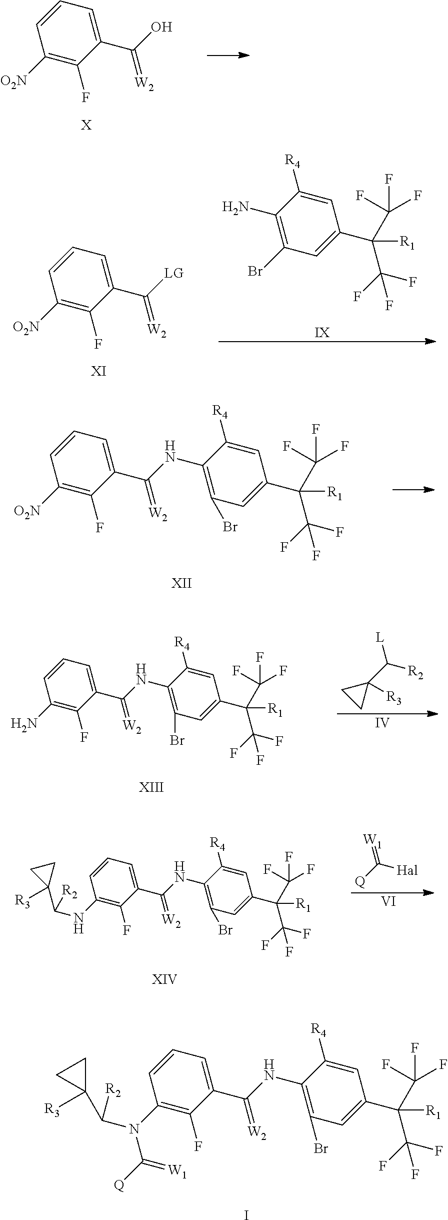

Formula X→Formula XI 2-(i):

A compound represented by the general formula XI having a leaving group can be prepared by a well-known method reacting a compound represented by the general formula X with thionyl chloride, oxalyl chloride, triphosgene or the like.

Formula XI+Formula IX→Formula XII 2-(ii):

By reacting a compound represented by the general formula XI with a compound represented by the general formula IX according to the conditions described in 1-(v), a compound represented by the general formula XII can be prepared.

Formula XII→Formula XIII 2-(iii):

An aromatic carboxamide derivative having an amino group represented by formula XIII can be derived from the aromatic carboxamide derivative having a nitro group represented by formula XII by means of a reduction reaction.

Such reduction is illustrated by a process using hydrogenation, a process using a metal compound (for example, stannous chloride) or a metal such as iron powder, zinc power and the like.

The hydrogenation reaction can be carried out in a suitable solvent in the presence of catalyst at atmospheric pressure or a higher pressure under a hydrogen atmosphere. Examples of the catalyst may include palladium catalysts such as palladium-carbon, cobalt catalysts, ruthenium catalysts, platinum catalysts and the like. Examples of the solvent may include alcohols such as methanol and ethanol; aromatic hydrocarbons such as benzene and toluene; chained or cyclic ethers such as ether and tetrahydrofuran; esters such as ethyl acetate.

Preferably, the hydrogenation reaction pressure can be appropriately selected within the range from 0.1 MPa to 10 MPa.

Preferably, the hydrogenation reaction temperature can be appropriately selected within the range from −20° C. to the boiling point of the solvent used, such as −20° C., 0° C., 15° C., 45° C., 75° C. or the boiling point, i.e., the reflux temperature of the solvent used.

Preferably, the hydrogenation reaction time can be appropriately selected within the range from half an hour to 48 hours.

Preferably, the process using a metal compound or a metal is conducted in any one of methanol, ethanol, ethyl acetate or the mixture of at least two thereof.

Preferably, the metal compound is stannous chloride and the metal is any one of iron powder, zinc power or a combination of at least two thereof.

Preferably, the reaction temperature using a metal compound or a metal can be appropriately selected within the range from −10° C. to the boiling point of the solvent used, such as −10° C., 20° C., 40° C., 80° C. or the boiling point, i.e., the reflux temperature of the solvent used.

Preferably, the reaction time using a metal compound or a metal can be appropriately selected within the range from half an hour to 48 hours.

Formula XIII+Formula IV→Formula XIV 2-(iv):

By reacting a compound represented by the general formula XIII with a compound represented by the general formula IV according to the conditions described in 1-(i), a compound represented by the general formula XIV can be prepared.

Formula XIV+Formula VI→Formula I 2-(v):

By reacting a compound represented by the general formula XIV with a compound represented by the general formula VI according to the conditions described in 1-(ii), a compound represented by the general formula I can be prepared.

### Preparation Method 3

The compounds of general formula I of this invention can be prepared by an alternative method shown below:

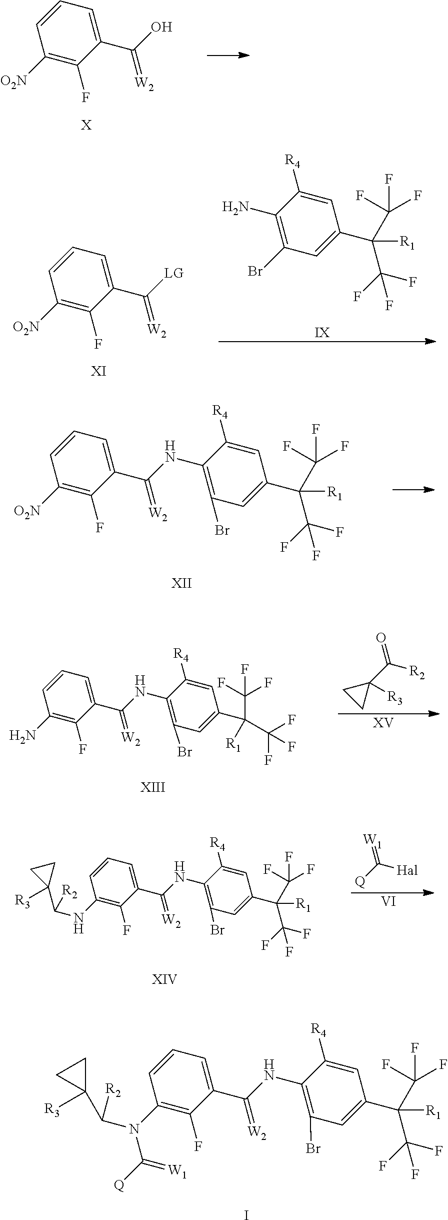

Formula X→Formula XI 3-(i):

By reacting a compound represented by the general formula X according to the conditions described in 2-(i), a compound represented by the general formula XI can be prepared.

Formula XI+Formula IX→Formula XII 3-(ii):

By reacting a compound represented by the general formula XI with a compound represented by the general formula IX according to the conditions described in 1-(v), a compound represented by the general formula XII can be prepared.

Formula XII→Formula XIII 3-(iii):

By reacting a compound represented by the general formula XII according to the conditions described in 2-(iii), a compound represented by the general formula XIII can be prepared.

Formula XIII+Formula XV→Formula XIV 3-(iv):

Preferably, the compound represented by Formula XIII can be suitably selected in the range of 0.5 to 2 molar equivalents based on the compound represented by Formula XV.

The process of the reaction 3-(iv) is illustrated by a process using an acid (organic bases and/or inorganic bases) and a reductant (borohydrides).

Preferably, examples of the organic acids include any one of formic acid, acetic acid, trifluoroacetic acid, methanesulfonic acid or a combination of at least two thereof.

Preferably, examples of the inorganic acids include any one of hydrochloric acid, phosphoric acid, sulfuric acid or a combination of at least two thereof.

Preferably, examples of the reductants include sodium borohydride, sodium cyanoborohydride or sodium triacetoxyborohydride.

Preferably, solvents of the reaction 3-(iv) include any one of dichloromethane, toluene, ethyl acetate, acetone, tetrahydrofuran, dioxane, N, N-dimethylformamide or a combination of at least two thereof.

Preferably, the reaction temperature of the reaction 3-(iv) can be appropriately selected within the range from room temperature to the boiling point of the solvent used, such as 25° C., 40° C., 60° C., 90° C. or the boiling point, i.e., the reflux temperature of the solvent used.

Preferably, the reaction time of 3-(iv) can be appropriately selected within the range from half an hour to 48 hour.

Formula XIV+Formula VI→Formula I 3-(v):

By reacting a compound represented by the general formula XIV with a compound represented by the general formula VI according to the conditions described in 1-(ii), a compound represented by the general formula I can be prepared.

On the other hand, this invention provides an intermediate representing by formula XIV for preparing amide compounds of formula I.

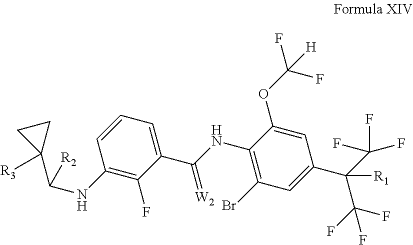

Wherein W₂, R₁, R₂ and R₃ have the same definition as the general formula I.

The preparation of intermediate XIV has been involved in the preparation method of the compounds of formula I above, and will not be repeated here.

Table 2 lists the representative compounds of intermediate XIV, but the present invention is not limited thereto.

TABLE 2

| No. | W₂ | R₁ | R₂ | R₃ | Appearance |
|---|---|---|---|---|---|
| 1. | O | F | H | H | Yellow oil |
| 2. | O | H | H | H | Brown oil |
| 3. | S | F | H | H | |
| 4. | S | H | H | H | |
| 5. | O | F | Me | H | |
| 6. | O | F | Me | Cl | |
| 7. | O | F | CH₂Cl | Cl | |
| 8. | O | F | CH₂F | H | |
| 9. | O | F | CH₂F | Cl | |
| 10. | O | F | c-Pr | H | |
| 11. | O | F | CF₃ | H | |

Furthermore, this invention provides tautomers, enantiomers, non-enantiomers or salts of amide compounds.

The tautomers, enantiomers, non-enantiomers or salts of amide derivatives have the same insecticidal activity as the amide derivatives, i.e., they have good insecticidal activity at low amount and quick-acting property.

Furthermore, this invention provides use of the amide compounds for controlling plant pests and nematodes in agriculture, forestry and horticulture.

The amide derivatives of this invention can effectively control pests of agriculture, forestry, horticulture, public health and nematodes, which are harmful to paddies, corns, wheats, potatoes, fruit trees, vegetables, other crops and flowering plants, etc.

The pests according to this invention contain lepidoptera, coleoptera, hemiptera, thysanoptera, diptera, orthoptera, homoptera, isoptera, hymenoptera, tetranychidaeand nematodes, mosquitoes, flies, ants, etc.

Preferably, the pests according to this invention contain as follows but this invention is not limited thereto: Helicoverpa armigera(Hubner), Plutella xylostella(Linnaeus), Spodoptera exigua(Hubner), Spodoptera litura(Fabricius), Pieris rapae (Linne), Chilo suppressalis(Walker), Tryporyza incertulas (Walker), Sesamia inferens (Walker), Spodoptera frugiperda (J. E. Smith), Cnaphalocrocis medinalis(Guenee), Chloethrips oryzae (Wil.), Frankliniella occidentalis(Pergande), Thrips fevas (Schrank), Thrips alliorum (Priesner), Myzus persicae (Sulzer), Aphis gossypii (Glover), Aphis craccivora (Koch), Aphis citricolavander Goot, Rhopalosiphum padi, Flea beetle, Stinkbug, Laodelphax striatellus, Nilaparvata lugens (Stal), Sogatella furcifera, Termites, Flies and Mosquitoes, Tetranychus cinnabarinus, Citrus red mite.

The compounds of this invention can be broadly applied in the following categories: vegetables such as cucumber, loofah, watermelon, melon, pumpkin, hanging melon, spinach, celery, kale, cabbage, gourd, pepper, eggplant, tomato, shallot, ginger, garlic, leek, lettuce, kidney bean, cowpea, broad bean, radish, carrot, potato, yam; cereals such as wheat, barley, corn, rice, sorghum; fruits such as apple, pear, banana, citrus, grape, lychee, mango; flowering plants such as peony, rose, flamingo flower, oil crops such as peanuts, soybeans, rapeseed, sunflower, sesame; sugar-yielding crops such as sugar beets, sugarcane; other crops such as strawberries, potatoes, sweet potatoes, tobacco and tea; horticulture, forestry, home and public areas, etc. The usable scope of the amide derivatives according to this invention is not limited to the categories listed above.

On the other aspect, this invention provides an insecticidal composition comprising active ingredient(s) and acceptable carrier in agriculture, wherein the active ingredient(s) are the amide compounds described above.

The composition of this invention can be used in form of a formulation, wherein the compounds represented by the general formula I are dissolved or dispersed in the carrier as active ingredients or they can be formulated to make them easier to disperse when they are used as pesticides

The present disclosure relates to insecticide compositions, which can be made into a variety of formulation forms, such as, a wettable powder, a suspension concentrate, an aqueous emulsion or an emulsifiable concentrate, etc.

The present disclosure is designed to solve the problems of the related fields such as agriculture, forestry, public health, etc.

Preferably, in the insecticide composition, the weight percentage of the active component is 1-99%, such as 1%, 10%, 20%, 35%, 55%, 75%, 95% or 99%.

Preferably, the carrier acceptable in pesticide science includes surfactants.

The surfactants in the present disclosure include ionic surfactants or nonionic surfactants.

The surfactants include emulsifiers, dispersants, or wetting agents. The emulsifiers in present disclosure include polyoxyethylene fatty acid ester, polyoxyethylene aliphatic alcohol ether, fatty amine polyoxyethylene ether and commercially available emulsifiers, such as pesticide emulsifier 2201B, 0203B, 100 #, 500 #, 600 #, 600-2 #, 1601, 2201, NP-10, NP-15, 507 #, OX-635, OX-622, OX-653, OX-667, 36 # and the like. The dispersants in present disclosure include sodium lignin sulfonate, nekal, calcium lignin sulfonate, methylnaphthalene sulfonate formaldehyde condensate and so on. The wetting agents researched in present disclosure include sodium lauryl sulfate, sodium dodecyl benzene sulfonate, sodium alkyl naphthalene sulfonate and the like.

Preferably, the carriers acceptable in pesticide science include solid carriers and/or liquid carriers.

Preferably, the solid carriers in present disclosure include natural or synthetic clays and silicates (for example, natural silica, diatomite); magnesium silicate (for example, talc); magnesium aluminum silicate (for example, kaolinite, kaolin, montmorillonite and mica); precipitated silica, calcium carbonate, light calcium carbonate, calcium sulfate, limestone, sodium sulfate; amine salt (for example, ammonium sulfate, hexamethylenediamine). The liquid carriers in present disclosure include water and organic solvents. When water is used as a solvent or diluent, organic solvents can also be used as additives or antifreeze additives. The suitable organic solvents in present disclosure include aromatic hydrocarbon (for example, benzene, xylene, toluene and the like); chlorinated hydrocarbon (for example, chlorobenzene, chloroethylene, trichloromethane, dichloromethane and the like); aliphatic hydrocarbon (for example, petroleum fractions, cyclohexane, light mineral oil and the like); alcohols (for example, isopropanol, butanol, glycol, glycerol and cyclohexanol and the like), their ethers and esters; ketones (for example, acetone, cyclohexanone); dimethylformamide and N-methylpyrrolidone.

During the preparation of the pesticide composition, the active ingredient(s) may be mixed with the liquid and/or solid carriers. Surfactants (such as emulsifiers, dispersants, stabilizers, wetting agents) and other auxiliaries (such as adhesives, defoaming agents, oxidants, etc.) may be added as well.

On the other aspect, this invention provides a method for controlling pests, wherein an effective amount of the amide compounds, or the tautomers, enantiomers, diasteromers or salts thereof, or the composition described above will be used to the pests to be controlled or to their habitat.

Preferably, the effective amount is from 7.5 g/ha to 1000 g/ha, such as 7.5 g/ha, 50 g/ha, 100 g/ha, 180 g/ha, 250 g/ha, 350 g/ha, 450 g/ha, 600 g/ha, 800 g/ha, or 1000 g/ha. More preferably, the effective amount is from 15 g/ha to 600 g/ha.

The composition of this invention can be used to the pests and their habitat in form of a formulation. The compounds represented by the general formula I are dissolved or dispersed in the carrier as an active ingredient or they can be formulated to make them easier to disperse when they are used as pesticides. These compounds can be formulated into various liquid formulations, emulsifiable concentrates, suspensions, aqueous suspensions, microemulsions, emulsions, aqueous emulsions, powder, wettable powder, soluble powder, granules, aqueous dispersible granules or capsule.

For certain applications, for example, in agriculture, one or more additional agents, such as insecticides, fungicides, herbicides, plant growth regulators or fertilizers, can be added into the insecticide composition of this invention, so as to obtain additional advantages and effects.

Comparing with the prior art, this invention has following benefits:

The amide derivatives of this invention are significantly effective for controlling the pests and nematodes in agriculture, forestry and public health. They have excellent insecticidal activity at low amount, which can be exerted after one day of application, and excellent insecticidal activity can be achieved on the third day, with good quick-acting property. The good insecticidal activity at low amount of the amide derivatives of this invention can reduce the damage of pesticide application to plant and human beings and the residue of pesticide, so they are more conducive to environmental protection. The methods for production are also simple and efficient, and the mass production can be easily realized. Thus the compounds and the compositions of this invention have a wide application prospect.

### DETAILED DESCRIPTION

Representative Examples of this invention will be described in the following Examples. Those skilled in the art should understand that the examples herein are only illustrative, and this invention is not limited thereto. Unless otherwise stated, compounds were dissolved in DMSO-d₆ and measured by Brucker 400 MHz spectrometer to obtain their ¹H NMR spectra, respectively. Chemical shifts were given in ppm relevant to a TMS standard. SGC represents silica gel column chromatography, PE represents petroleum ether, EA represents ethyl acetate in the following examples.

### PREPARATION EXAMPLES

### Example 1: Preparation of N-(2-bromo-4-(perfluoropropan-2-yl)-6-(difluoromethoxy) phenyl)-3-(N-(cyclopropylmethyl)benzamido)-2-fluorobenzamide (Compound No. 1)

### Step 1: N-(2-bromo-4-(perfluoropropan-2-yl)-6-(difluoromethoxy)phenyl)-2-fluoro-3-nitrobenzamide

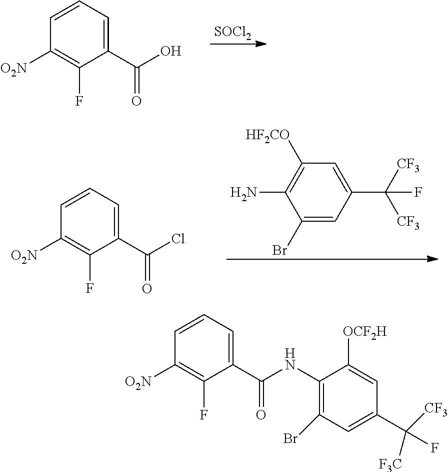

Thionyl chloride (25.7 g, 216.1 mmol) was added to 2-fluoro-3-nitrobenzoic acid (11.1 g, 59.85 mmol) in toluene (30 mL), and the mixture was heated and refluxed for 2 hours. The solvent was removed by distillation to get the coarse product 2-fluoro-3-nitrobenzoyl chloride. To 2-fluoro-3-nitrobenzoyl chloride was added 2-bromo-6-(difluoromethoxy)-4-(perfluoropropan-2-yl)aniline (20.25 g, 58.85 mmol), N, N-diisopropylethylamine (12.89 g, 99.75 mmol) and N, N-dimethylpyridin-4-amine (2.44 g, 19.95 mmol). The mixture was stirred at 110° C. for 8 hours. TLC showed the reaction was completed. The reaction mixture was diluted with H₂O (100 mL) and extracted with EA (200 mL). The organic layer was washed with saturated brine, dried over anhydrous magnesium sulphate and evaporated under reduced pressure. The residue was purified by SGC (eluent: PE:EA=5:1) to obtain 10.4 g (yield 30.32%) of the target compound.

¹H NMR: 10.79 (s, 1H), 8.36 (t, J=8.0 Hz, 1H), 8.02 (t, J=8.0 Hz, 1H), 7.93 (s, 1H), 7.62 (t, J=8.0 Hz, 2H), 7.40 (t, J=72 Hz, 1H).

### Step 2: Preparation of 3-amino-N-(2-bromo-6-(difluoromethoxy)-4-(perfluoropropan-2-yl) phenyl)-2-fluorobenzamide

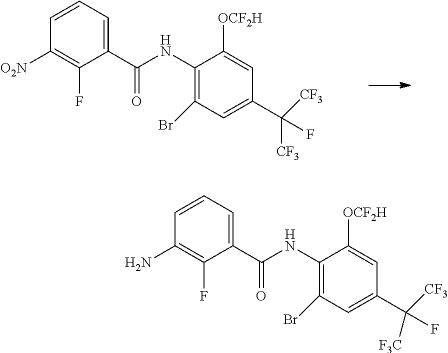

To the solution of N-(2-bromo-4-(perfluoropropan-2-yl)-6-(difluoromethoxy)phenyl)-2-fluoro-3-nitrobenzamide (10.4 g, 18.15 mmol) in anhydrous EtOH (50 mL) was added tin(II) chloride dihydrate (16.37 g, 72.58 mmol) and concentrated hydrochloric acid (0.5 mL). Then the mixture was heated and refluxed for 3 hours. TLC showed the reaction was finished. After the solvent was removed by distillation, the pH of the mixture was adjusted by 10% sodium hydroxide solution to 12. The reaction mixture was extracted with EA (200 mL). The organic layer was washed with saturated brine and dried over anhydrous magnesium sulphate. The solvent was evaporated under reduced pressure and the obtained residue was purified by SGC (eluent: PE:EA=5:1) to obtain 7.4 g (yield 75.05%) of the target compound as brown oil.

¹H NMR: 10.20 (s, 1H), 7.89 (s, 1H), 7.53 (s, 1H), 7.32 (t, J=72.0 Hz, 1H), 7.03-6.89 (m, 2H), 6.80 (t, J=6.7 Hz, 1H), 5.39 (s, 2H).

### Step 3: Preparation of N-(2-bromo-6-(difluoromethoxy)-4-(perfluoropropan-2-yl)phenyl)-3-((cyclopropylmethyl)amino)-2-fluorobenzamide

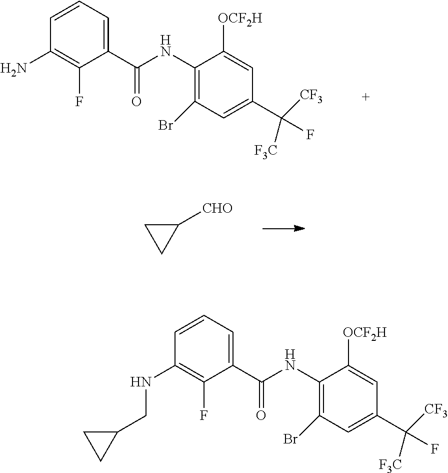

To the solution of 3-amino-N-(2-bromo-6-(difluoromethoxy)-4-(perfluoropropan-2-yl) phenyl)-2-fluorobenzamide (3.0 g, 5.53 mmol) in anhydrous 1,2-dichloroethane (30 mL) was added cyclopropanecarbaldehyde (0.37 g, 5.08 mmol) and trifluoroacetic acid (7.78 g, 33.14 mmol). Then the reaction mixture was stirred at room temperature for 10 mins. Sodium triacetoxyborohydride (3.51 g, 16.57 mmol) was added to the mixture. TLC showed the reaction was finished. After the solvent was removed by distillation, the pH of the mixture was adjusted by saturated sodium bicarbonate aqueous solution to 8. The reaction mixture was extracted with dichloromethane (20 mL). The organic layer was washed with saturated brine and dried over anhydrous magnesium sulphate. The solvent was evaporated under reduced pressure and the obtained residue was purified by SGC (eluent: PE:EA=10:1) to obtain 2.47 g (yield 75%) of the target compound as yellow oil.

¹H NMR: 10.01 (s, 1H), 7.66 (s, 1H), 7.30 (s, 1H), 7.09 (t, J=72.0 Hz, 1H), 6.85 (t, J=7.8 Hz, 1H), 6.69 (t, J=7.7 Hz, 1H), 6.56 (t, J=6.2 Hz, 1H), 5.47 (s, 1H), 2.79 (t, J=5.7 Hz, 2H), 0.90-0.80 (m, 1H), 0.24-0.18 (m, 2H), 0.01 (q, J=4.9 Hz, 2H).

### Step 4: Preparation of N-(2-bromo-4-(perfluoropropan-2-yl)-6-(difluoromethoxy)phenyl)-3-(N-(cyclopropylmethyl)benzamido)-2-fluorobenzamide

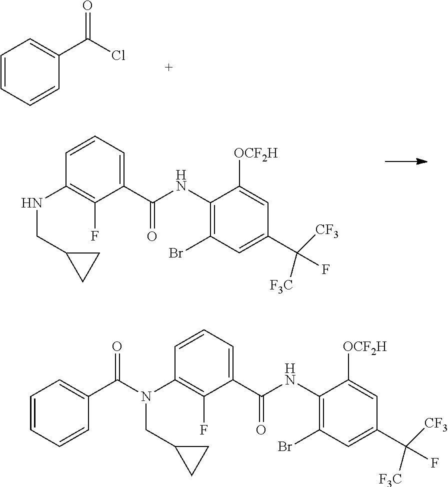

To the solution of N-(2-bromo-6-(difluoromethoxy)-4-(perfluoropropan-2-yl)phenyl)-3-((cyclopropylmethyl)amino)-2-fluorobenzamide (0.30 g, 0.50 mmol) in anhydrous tetrahydrofuran (5 mL) was added benzoyl chloride (77 mg, 0.55 mmol) and pyridine (79 mg, 1.00 mmol). The mixture was stirred at 80° C. for 4 hours. TLC showed the reaction was completed. The reaction mixture was extracted with EA (40 mL), washed with 2M HCl (5 mL) and saturated sodium bicarbonate aqueous solution (30 mL), dried over anhydrous magnesium sulphate and evaporated under reduced pressure. The residue was purified by SGC (eluent: PE:EA=8:1) to obtain 0.18 g (yield 52.63%) of the target compound.

Compound No. 1: ¹H NMR: 10.32 (s, 1H), 7.91 (s, 1H), 7.64-7.50 (m, 4H), 7.33-7.15 (m, 6H), 3.70 (d, J=76.0 Hz, 2H), 1.05-1.03 (m, 1H), 0.41 (d, J=8.0 Hz, 2H), 0.09 (br s, 2H).

### Example 2: Preparation of N-(2-bromo-6-(difluoromethoxy)-4-(perfluoropropan-2-yl)phenyl)-3-(N-(cyclopropylmethyl)-4-fluorobenzamido)-2-fluorobenzamide (Compound No. 31)

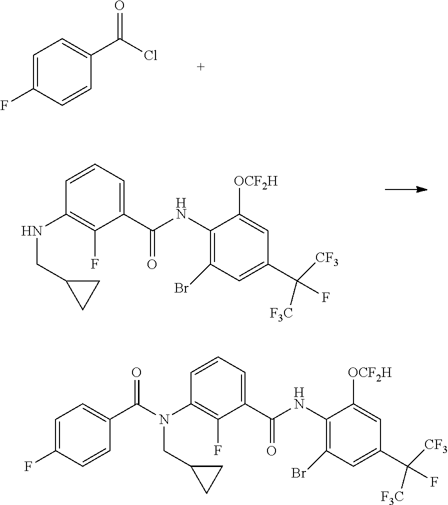

To the solution of N-(2-bromo-6-(difluoromethoxy)-4-(perfluoropropan-2-yl)phenyl)-3 ((cyclopropylmethyl)amino)-2-fluorobenzamide (0.30 g, 0.50 mmol) in anhydrous tetrahydrofuran (5 mL) was added 4-fluorobenzoyl chloride (87 mg, 0.55 mmol) and pyridine (79 mg, 1.00 mmol). The mixture was stirred at 80° C. for 4 hours. TLC showed the reaction was completed. The reaction mixture was extracted with EA (40 mL), washed with 2M HCl (5 mL) and saturated sodium bicarbonate aqueous solution (30 mL), dried over anhydrous magnesium sulphate and evaporated under reduced pressure. The residue was purified by SGC (eluent: PE:EA=8:1) to obtain 0.054 g (yield 15.01%) of the target compound.

Compound No. 31: ¹H NMR: 10.32 (s, 1H), 7.90 (s, 1H), 7.67-7.51 (m, 4H), 7.38-7.33 (m, 3H), 7.15-7.09 (m, 2H), 3.70 (d, J=20.0 Hz, 2H), 1.06-1.01 (m, 1H), 0.41 (d, J=8.0 Hz, 2H), 0.09 (br s, 2H).

### Example 3: Preparation of N-(2-bromo-6-(difluoromethoxy)-4-(1,1,1,3,3,3-hexafluoropropan-2-yl)phenyl)-3-(4-cyano-N-(cyclopropylmethyl)benzamido)-2-fluorobenzamide (Compound No. 26)

### Step 1: Preparation of N-(2-bromo-6-(difluoromethoxy)-4-(1,1,1,3,3,3-hexafluoropropan-2-yl)phenyl)-2-fluoro-3-nitrobenzamide

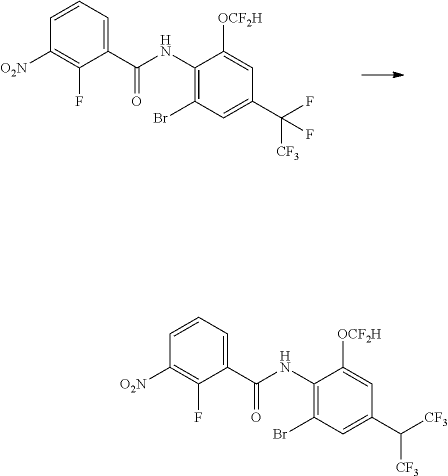

To the solution of N-(2-bromo-6-(difluoromethoxy)-4-(perfluoropropan-2-yl)phenyl)-2-fluoro-3-nitrobenzamide (2.29 g, 4.0 mmol) in anhydrous dimethyl sulfoxide (20 mL) was added sodium borohydride (300 mg, 8.0 mmol). Then the mixture was heated at 60° C. for 4 hours. TLC showed the reaction was finished. The reaction mixture was diluted with H₂O (50 mL) and extracted with EA (50 mL). The organic layer was washed with saturated brine, dried over anhydrous magnesium sulphate and evaporated under reduced pressure. The residue was purified by SGC (eluent: PE:EA=10:1) to obtain 1.10 g (yield 49.55%) of the target compound as yellow oil.

¹H NMR: 10.83 (s, 1H), 8.82 (s, 1H), 8.52 (d, J=8.0 Hz, 1H), 8.43 (d, J=8.0 Hz, 1H), 7.94 (s, 1H), 7.90 (t, J=8.0 Hz, 1H), 7.59 (s, 1H), 7.38 (t, J=72 Hz, 1H).

### Step 2: Preparation of 3-amino-N-(2-bromo-6-(difluoromethoxy)-4-(1,1,1,3,3,3-hexafluoropropan-2-yl)phenyl)-2-fluorobenzamide

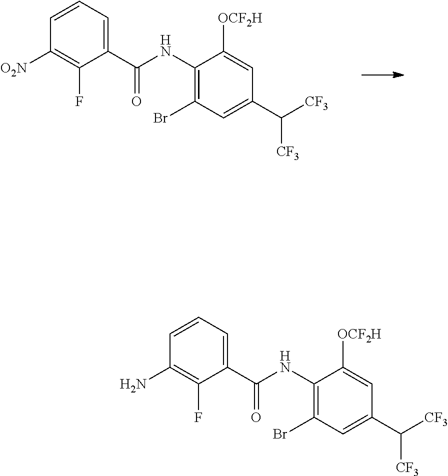

To the solution of N-(2-bromo-6-(difluoromethoxy)-4-(1,1,1,3,3,3-hexafluoropropan-2-yl) phenyl)-2-fluoro-3-nitrobenzamide (1.1 g, 1.97 mmol) in anhydrous EtOH (20 mL) was added tin(II) chloride dihydrate (1.70 g, 7.90 mmol) and concentrated hydrochloric acid (0.2 mL). Then the mixture was heated and refluxed for 3 hours. TLC showed the reaction was finished. After the solvent was removed by distillation, the pH of the mixture was adjusted by 10% sodium hydroxide solution to 12. The reaction mixture was extracted with EA (50 mL). The organic layer was washed with saturated brine and dried over anhydrous magnesium sulphate. The solvent was evaporated under reduced pressure and the obtained residue was purified by SGC (eluent: PE:EA=5:1) to obtain 0.8 g (yield 76.92%) of the target compound as yellow solid.

¹H NMR: 10.10 (s, 1H), 7.89 (s, 1H), 7.52 (s, 1H), 7.31 (t, J=72 Hz, 1H), 7.19-7.10 (m, 3H), 6.78 (d, J=8.0 Hz, 1H), 5.36 (s, 2H).

### Step 3: Preparation of N-(2-bromo-6-(difluoromethoxy)-4-(1,1,1,3,3,3-hexafluoropropan-2-yl)phenyl)-3-((cyclopropylmethyl)amino)-2-fluorobenzamide

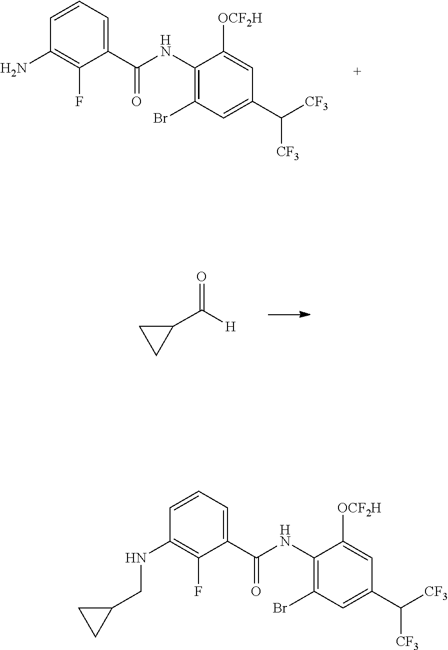

To the solution of 3-amino-N-(2-bromo-6-(difluoromethoxy)-4-(1,1,1,3,3,3-hexafluoropropan-2-yl)phenyl)-2-fluorobenzamide (0.8 g, 1.52 mmol) in anhydrous 1,2-dichloroethane (20 mL) was added cyclopropanecarbaldehyde (99 mg, 1.37 mmol) and trifluoroacetic acid (1.04 g, 9.12 mmol). Then the reaction mixture was stirred at room temperature for 10 mins. Sodium triacetoxyborohydride (0.96 g, 4.56 mmol) was added to the mixture. TLC showed the reaction was finished. After the solvent was removed by distillation, the pH of the mixture was adjusted by saturated sodium bicarbonate aqueous solution to 8. The reaction mixture was extracted with dichloromethane (20 mL). The organic layer was washed with saturated brine and dried over anhydrous magnesium sulphate. The solvent was evaporated under reduced pressure and the obtained residue was purified by SGC (eluent: PE:EA=10:1) to obtain 0.60 g (yield 68.18%) of the target compound as brown oil.

### Step 4: Preparation of N-(2-bromo-6-(difluoromethoxy)-4-(1,1,1,3,3,3-hexafluoropropan-2-yl) phenyl)-3-(4-cyano-N-(cyclopropylmethyl)benzamido)-2-fluorobenzamide

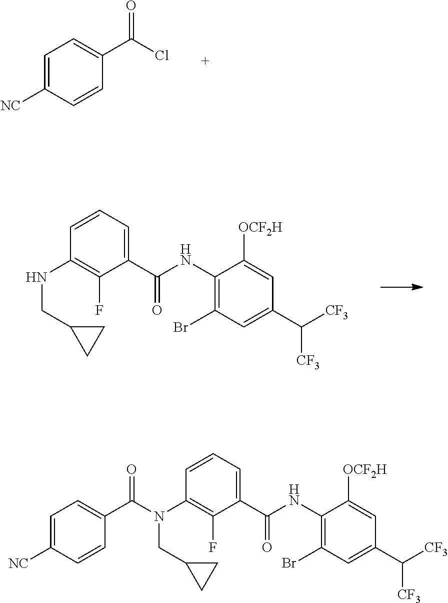

To the solution of N-(2-bromo-6-(difluoromethoxy)-4-(1,1,1,3,3,3-hexafluoropropan-2-yl) phenyl)-3-((cyclopropylmethyl)amino)-2-fluorobenzamide (0.20 g, 0.34 mmol) in toluene (5 mL) was added 4-cyanobenzoyl chloride (83 mg, 0.52 mmol) and N, N-diisopropylethylamine (66 mg, 0.52 mmol). The mixture was stirred at reflux for 4 hours. The reaction mixture was diluted with H₂O (20 mL) and extracted with EA (20 mL). The organic layer was washed with saturated brine, dried over anhydrous magnesium sulphate and evaporated under reduced pressure. The residue was purified by SGC (eluent: PE:EA=6:1) to obtain 0.15 g (yield 62.31%) of the target compound as white solid.

Compound No. 26: ¹H NMR: 10.32 (s, 1H), 7.92 (s, 1H), 7.81-7.76 (m, 2H), 7.72 (d, J=8.0 Hz, 2H), 7.54 (s, 1H), 7.51-7.47 (m, 3H), 7.31 (4, J=74.4 Hz, 2H), 3.79 (d, J=6.4 Hz, 2H), 1.07-0.99 (m, 1H), 0.45-0.41 (m, 2H), 0.16 (br s, 2H).

### Example 4: Preparation of N-(3-((2-bromo-4-(perfluoropropan-2-yl)-6-(trifluoromethyl) phenyl)carbamoyl)-2-fluorophenyl)-N-(cyclopropylmethyl)-6-fluoronicotinamide (Compound No. 106)

### Step 1: Preparation of 2-fluoro-3-nitrobenzoyl chloride

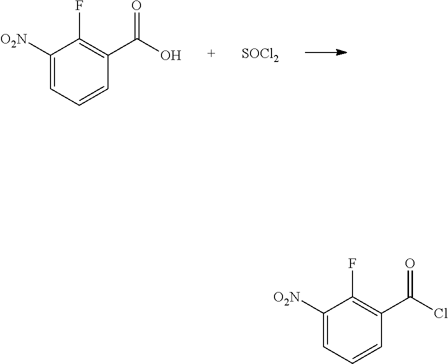 [SMILES: O=C(O)c1cccc([N+](=O)[O-])c1F ; "SOCl2" ; O=C(Cl)c1cccc([N+](=O)[O-])c1F]

Thionyl chloride (54.00 g, 455.64 mmol) was added to 2-fluoro-3-nitrobenzoic acid (16.87 g, 91.16 mmol) in toluene (200 mL), and the mixture was heated and refluxed for 2 hours. The solvent was removed by distillation to get the coarse product 2-fluoro-3-nitrobenzoyl chloride.

### Step 2: Preparation of N-(2-bromo-4-(perfluoropropan-2-yl)-6-(trifluoromethyl)phenyl)-2-fluoro-3-nitrobenzamide

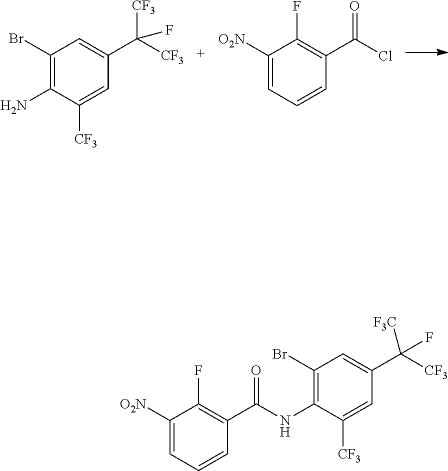 [SMILES: Nc1c(Br)cc(C(F)(C(F)(F)F)C(F)(F)F)cc1C(F)(F)F ; O=C(Cl)c1cccc([N+](=O)[O-])c1F ; O=C(Nc1c(Br)cc(C(F)(C(F)(F)F)C(F)(F)F)cc1C(F)(F)F)c1cccc([N+](=O)[O-])c1F]

To 2-fluoro-3-nitrobenzoyl chloride was added 2-bromo-4-(perfluoropropan-2-yl)-6-(trifluoromethyl)aniline (31.00 g, 75.97 mmol), N, N-diisopropylethylamine (19.64 g, 151.94 mmol) and N,N-dimethylpyridin-4-amine (3.71 g, 30.39 mmol). The mixture was stirred at 100° C. TLC showed the reaction was completed. The reaction mixture was diluted with H₂O (100 mL) and extracted with EA (100 mL). The organic layer was washed with saturated brine, dried over anhydrous magnesium sulphate and evaporated under reduced pressure. The residue was purified by SGC (eluent: PE:EA=4:1) to obtain 21.82 g (yield 50.00%) of the target compound as yellow oil.

### Step 3: Preparation of 3-amino-N-(2-bromo-4-(perfluoropropan-2-yl)-6-(trifluoromethyl) phenyl)-2-fluorobenzamide

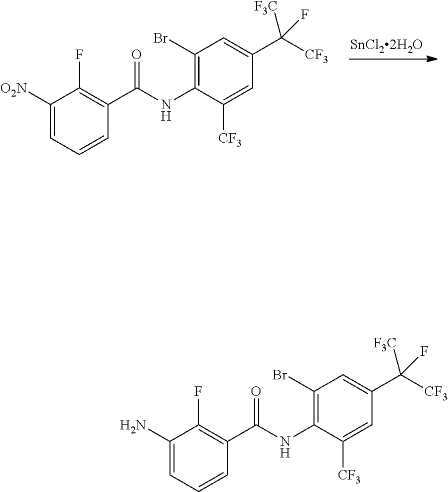 [SMILES: O=C(Nc1c(Br)cc(C(F)(C(F)(F)F)C(F)(F)F)cc1C(F)(F)F)c1cccc([N+](=O)[O-])c1F ; Nc1cccc(C(=O)Nc2c(Br)cc(C(F)(C(F)(F)F)C(F)(F)F)cc2C(F)(F)F)c1F]

To the solution of N-(2-bromo-4-(perfluoropropan-2-yl)-6-(trifluoromethyl)phenyl)-2-fluoro-3-nitrobenzamide (21.82 g, 37.94 mmol) in anhydrous EtOH (200 mL) was added tin(II) chloride dihydrate (34.24 g, 151.76 mmol) and concentrated hydrochloric acid (3 mL). Then the mixture was heated and refluxed for 2 hours. TLC showed the reaction was finished. After the solvent was removed by distillation, the pH of the mixture was adjusted by 10% sodium hydroxide solution to 10. The reaction mixture was extracted with EA (200 mL). The organic layer was washed with saturated brine and dried over anhydrous magnesium sulphate. The solvent was evaporated under reduced pressure and the obtained residue was purified by SGC (eluent: PE:EA=4:1) to obtain 18.08 g (yield 87.40%) of the target compound as yellow solid.

### Step 4: Preparation of N-(2-bromo-4-(perfluoropropan-2-yl)-6-(trifluoromethyl)phenyl)-3-((cyclopropylmethyl)amino)-2-fluorobenzamide

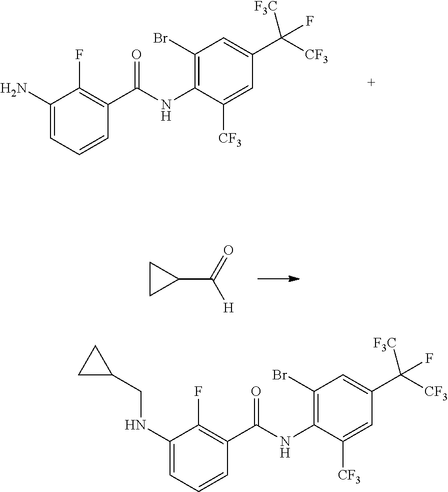 [SMILES: Nc1cccc(C(=O)Nc2c(Br)cc(C(F)(C(F)(F)F)C(F)(F)F)cc2C(F)(F)F)c1F ; O=CC1CC1 ; O=C(Nc1c(Br)cc(C(F)(C(F)(F)F)C(F)(F)F)cc1C(F)(F)F)c1cccc(NCC2CC2)c1F]

To the solution of 3-amino-N-(2-bromo-4-(perfluoropropan-2-yl)-6-(trifluoromethyl)phenyl)-2-fluorobenzamide (5 g, 9.19 mmol) in 1,2-dichloroethane (20 mL) was added cyclopropanecarbaldehyde (580 mg, 8.27 mmol) and trifluoroacetic acid (6.27 g, 55.02 mmol). Then the reaction mixture was stirred at room temperature for 10 mins. Sodium triacetoxyborohydride (5.83 g, 27.51 mmol) was added to the mixture. TLC showed the reaction was finished. After the solvent was removed by distillation, the pH of the mixture was adjusted by saturated sodium bicarbonate aqueous solution to 8. The reaction mixture was extracted with dichloromethane (20 mL). The organic layer was washed with saturated brine and dried over anhydrous magnesium sulphate. The solvent was evaporated under reduced pressure and the obtained residue was purified by SGC (eluent: PE:EA=20:1) to obtain 3.94 g (yield 71.8%) of the target compound as brown oil.

### Step 5: Preparation of N-(3-((2-bromo-4-(perfluoropropan-2-yl)-6-(trifluoromethyl)phenyl) carbamoyl)-2-fluorophenyl)-N-(cyclopropylmethyl)-6-fluoronicotinamide

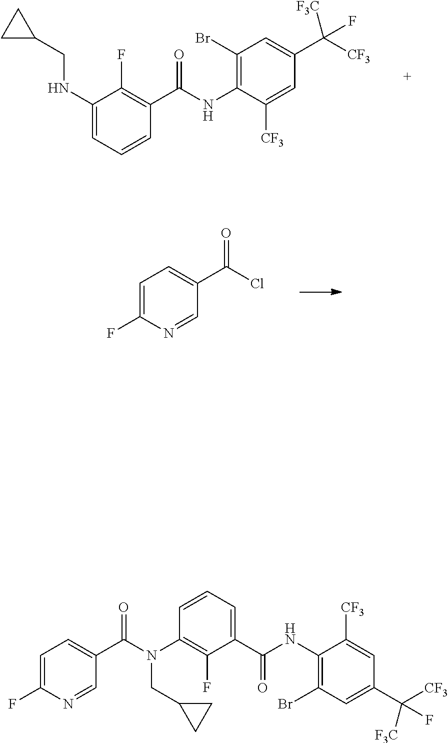 [SMILES: O=C(Nc1c(Br)cc(C(F)(C(F)(F)F)C(F)(F)F)cc1C(F)(F)F)c1cccc(NCC2CC2)c1F ; O=C(Cl)c1ccc(F)nc1 ; O=C(Nc1c(Br)cc(C(F)(C(F)(F)F)C(F)(F)F)cc1C(F)(F)F)c1cccc(N(CC2CC2)C(=O)c2ccc(F)nc2)c1F]

To the solution of N-(2-bromo-4-(perfluoropropan-2-yl)-6-(trifluoromethyl)phenyl)-3-((cyclopropylmethyl)amino)-2-fluorobenzamide (300 mg, 0.50 mmol) in toluene (5 mL) was added 6-fluoronicotinoyl chloride (87.86 mg, 0.55 mmol) and N, N-diisopropylethylamine (97.06 mg, 0.75 mmol). The mixture was stirred at 110° C. for 4 hours. TLC showed the reaction was completed. The reaction mixture was diluted with H₂O (10 mL) and extracted with EA (20 mL). The organic layer was washed with saturated brine, dried over anhydrous magnesium sulphate and evaporated under reduced pressure. The residue was purified by SGC (eluent: PE:EA=4:1) to obtain 145 mg (yield 40.09%) of the target compound as yellow solid.

Compound No. 106: ¹H NMR: 10.62 (s, 1H), 8.42 (s, 1H), 8.15 (s, 1H), 7.95 (s, 2H), 7.78 (t, J=7.1 Hz, 1H), 7.62 (s, 1H), 7.39 (t, J=7.8 Hz, 1H), 7.12 (s, 1H), 3.74 (d, J=45.7 Hz, 2H), 1.03 (br s, 1H), 0.42 (d, J=6.4 Hz, 2H), 0.11 (d, J=27.7 Hz, 2H).

### Example 5: Preparation of N-(3-((2-bromo-6-(difluoromethoxy)-4-(perfluoropropan-2-yl) phenyl)carbamoyl)-2-fluorophenyl)-N-(cyclopropylmethyl)-6-fluoronicotinamide (Compound No. 118)

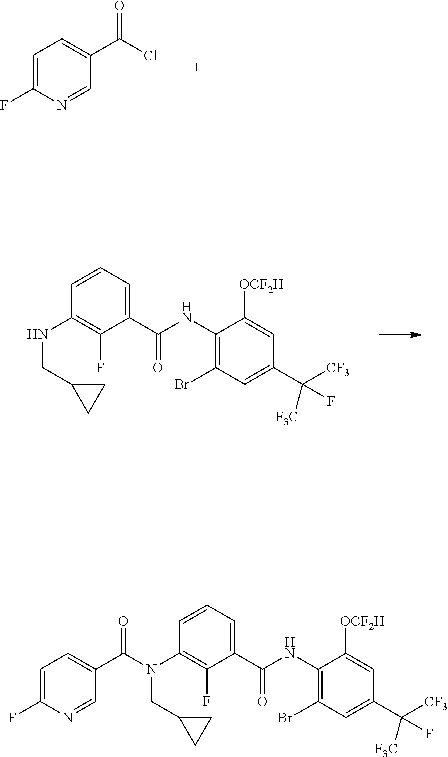

To the solution of N-(2-bromo-6-(difluoromethoxy)-4-(perfluoropropan-2-yl)phenyl)-3-((cyclopropylmethyl)amino)-2-fluorobenzamide (0.30 g, 0.50 mmol) in toluene (5 mL) was added 6-fluoronicotinoyl chloride (96 mg, 0.60 mmol) and N, N-diisopropylethylamine (97 mg, 0.75 mmol). The mixture was stirred at reflux for 4 hours. TLC showed the reaction was completed. The reaction mixture was extracted with EA (40 mL), washed with 2M HCl (5 mL), saturated sodium bicarbonate aqueous solution (30 mL), dried over anhydrous magnesium sulphate and evaporated under reduced pressure. The residue was purified by SGC (eluent: PE:EA=5:1) to obtain 89 mg (yield 25.63%) of the target compound.

Compound No. 118: ¹H NMR: 10.36 (s, 1H), 8.15 (s, 1H), 7.94 (s, 1H), 7.90 (s, 1H), 7.75 (t, J=8.0 Hz, 1H), 7.60 (s, 1H), 7.54 (s, 1H), 7.36 (t, J=8.0 Hz, 1H), 7.32 (t, J=76.0 Hz, 1H), 7.14-7.10 (m, 1H), 3.73 (br s, 2H), 1.06-1.00 (m, 1H), 0.42 (d, J=8.0 Hz, 2H), 0.12 (d, J=20.0 Hz, 2H).

### Example 6: Preparation of N-(3-((2-bromo-4-(perfluoropropan-2-yl)-6-(trifluoromethyl) phenyl)carbamoyl)-2-fluorophenyl)-2-chloro-N-(1-cyclopropylethyl)isonicotinamide (Compound No. 124)

### Step 1: Preparation of meth 13-((1-cyclopropylethyl)amino)-2-fluorobenzoate

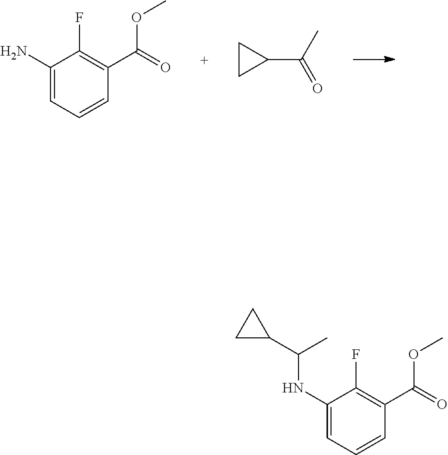

To the solution of methyl 3-amino-2-fluorobenzoate (2.00 g, 11.82 mmol) in 1,2-dichloroethane (65 mL) was added 1-cyclopropylethan-1-one (2.98 g, 35.47 mmol), trifluoroacetic acid (8.08 g, 70.92 mmol) and sodium triacetoxyborohydride (7.51 g, 35.47 mmol) was added to the mixture. The mixture was stirred at 45° C. for 1 hour. TLC showed the reaction was finished. After the solvent was removed by distillation, the pH of the mixture was adjusted by saturated sodium bicarbonate aqueous solution (50 mL) to 8. The reaction mixture was extracted with dichloromethane (80 mL). The organic layer was washed with saturated brine and dried over anhydrous magnesium sulphate. The solvent was evaporated under reduced pressure and the obtained residue was purified by SGC (eluent: PE:EA=10:1) to obtain 2.50 g (yield 89.11%) of the target compound as colorless oil.

### Step 2: Preparation of Methyl 3-(2-chloro-N-(1-cyclopropylethyl)isonicotinamido)-2-fluorobenzoate

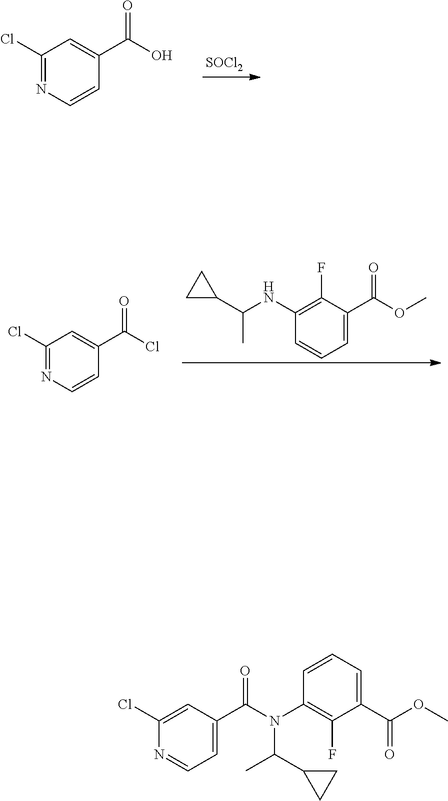

Thionyl chloride (4.93 g, 44.25 mmol) was added to 2-chloroisonicotinic acid (1.39 g, 8.85 mmol) in toluene (15 mL), and the mixture was heated and refluxed for 2 hours. After the solvent was removed by distillation, the coarse 2-chloroisonicotinoyl chloride in THF (5 mL) was used for the next step without further purification. To the solution of methyl 3-((1-cyclopropylethyl)amino)-2-fluorobenzoate (2.00 g, 8.43 mmol) in anhydrous THF (80 mL) was added triethylamine (0.90 g, 8.93 mmol) and 2-chloroisonicotinoyl chloride. The mixture was stirred at 80° C. for 6 hours. TLC showed the reaction was finished. The reaction mixture was diluted with H₂O (80 mL) and extracted with EA (100 mL). The organic layer was washed with saturated brine, dried over anhydrous magnesium sulphate and evaporated under reduced pressure. The residue was purified by SGC (eluent: PE:EA=10:1) to obtain 1.93 g (yield 60.89%) of the target compound as yellow solid.

### Step 3: Preparation of 3-(2-chloro-N-(1-cyclopropylethyl)isonicotinamido)-2-fluorobenzoic acid

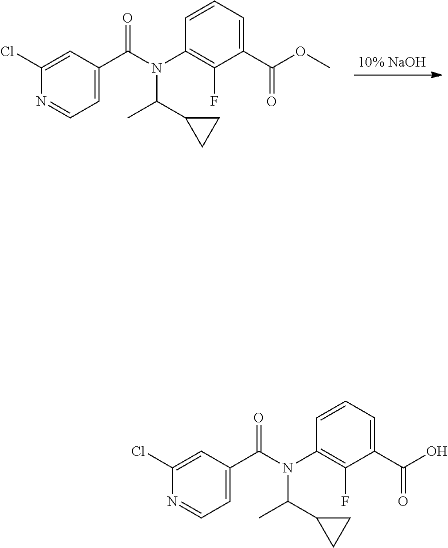 [SMILES: COC(=O)c1cccc(N(C(=O)c2ccnc(Cl)c2)C(C)C2CC2)c1F ; CC(C1CC1)N(C(=O)c1ccnc(Cl)c1)c1cccc(C(=O)O)c1F]

Methyl 3-(N-(1-cyclopropylethyl)-2-chloro isonicotinamido)-2-fluorobenzoate (1.50 g, 3.98 mmol) was dissolved in methanol (15 mL), 10% sodium hydroxide aqueous solution (6.4 mL) was added and the reaction mixture was stirred at room temperature for 2 hours. TLC showed the reaction was completed. After the solvent was removed by distillation, the coarse product was dissolved in H₂O (30 mL) and extracted with ethyl acetate (50 mL). The pH of the aqueous phase was acidified by the addition of 2M hydrochloric acid to 3 and extracted with ethyl acetate (40 mL). The organic layer was washed with saturated brine, dried over anhydrous magnesium sulphate and evaporated under reduced pressure to obtain 1.20 g (yield 83.09%) of the target compound.

### Step 4: Preparation of N-(3-((2-bromo-4-(perfluoropropan-2-yl)-6-(trifluoromethyl)phenyl) carbamoyl)-2-fluorophenyl)-2-chloro-N-(1-cyclopropylethyl)isonicotinamide

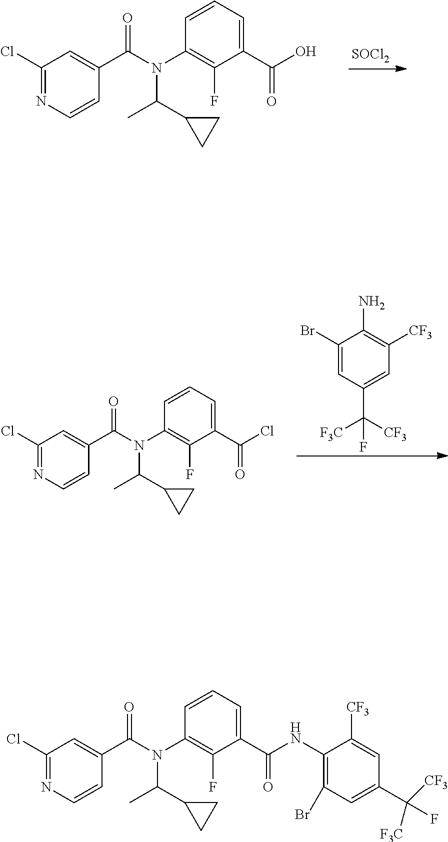

To the solution of 3-(N-(1-cyclopropylethyl)-2-chloroisonicotinamido)-2-fluorobenzoic acid (0.51 g, 1.40 mmol) in toluene (6 mL) was added thionyl chloride (0.73 g, 7.00 mmol). Then the mixture was heated and refluxed for 2 hours. After the solvent was removed by distillation, the coarse 3-(2-chloro-N-(1-cyclopropylethyl)isonicotinamido)-2-fluorobenzoyl chloride in THF (3 mL) was used for the next step without further purification. To 2-bromo-4-(perfluoropropan-2-yl)-6-(trifluoromethyl)aniline (0.52 g, 1.27 mmol) was added N, N-diisopropylethylamine (0.30 g, 2.55 mmol), N,N-dimethylpyridin-4-amine (62.28 mg, 509.76 μmol) and 3-(2-chloro-N-(1-cyclopropylethyl)isonicotinamido)-2-fluorobenzoyl chloride. The mixture was stirred at 110° C. for 2-3 hours. TLC showed the reaction was completed. The reaction mixture was diluted with H₂O (40 mL) and extracted with EA (60 mL). The organic layer was washed with saturated brine, dried over anhydrous magnesium sulphate and evaporated under reduced pressure. The residue was purified by SGC (eluent: PE:EA=4:1) to obtain 0.32 g (yield 33.25%) of the target compound as yellow solid.

Compound No. 124: ¹H NMR: 10.62 (d, J=28.4 Hz, 1H), 8.43 (s, 1H), 8.28 (d, J=4.8 Hz, 1H), 7.96 (s, 1H), 7.81 (dt, J=22.8, 7.1 Hz, 1H), 7.65 (s, 1H), 7.43-7.33 (m, 2H), 7.31-7.20 (m, 1H), 4.06 (br s, 1H), 1.40 (d, J=6.5 Hz, 1H), 1.24 (s, 3H), 0.60 (d, J=7.6 Hz, 2H), 0.41 (d, J=3.6 Hz, 2H) (m, 1H), 0.41 (d, J=8.0 Hz, 2H), 0.09 (br s, 2H).

### Example 7: Preparation of N-(3-((2-bromo-4-(1,1,1,3,3,3-hexafluoropropan-2-yl)-6-(trifluoromethyl)phenyl)carbamoyl)-2-fluorophenyl)-N-(cyclopropylmethyl)nicotinamide (Compund No. 156)

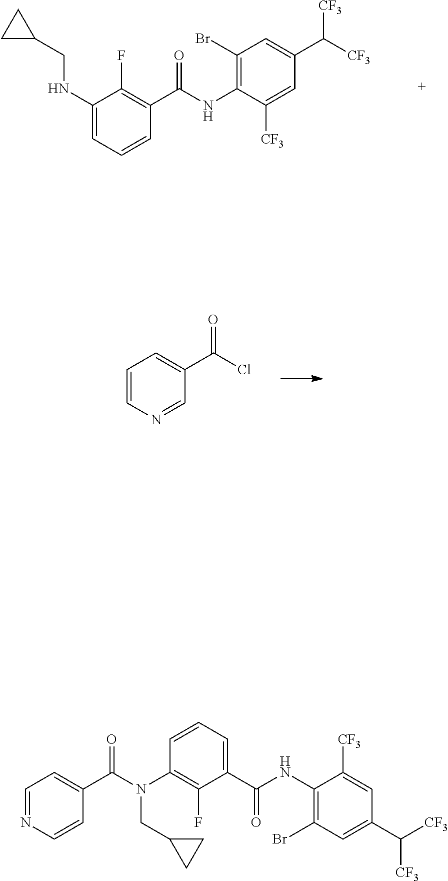 [SMILES: O=C(Nc1c(Br)cc(C(C(F)(F)F)C(F)(F)F)cc1C(F)(F)F)c1cccc(NCC2CC2)c1F ; O=C(Cl)c1cccnc1 ; O=C(Nc1c(Br)cc(C(C(F)(F)F)C(F)(F)F)cc1C(F)(F)F)c1cccc(N(CC2CC2)C(=O)c2ccncc2)c1F]

To the solution of N-(2-bromo-4-(1,1,1,3,3,3-hexafluoropropan-2-yl)-6-(trifluoromethyl) phenyl)-3-((cyclopropylmethyl)amino)-2-fluorobenzamide (200 mg, 0.34 mmol) in toluene (5 mL) was added nicotinoyl chloride (58 mg, 0.41 mmol) and N, N-diisopropylethylamine (89 mg, 0.69 mmol). The mixture was stirred at 110° C. TLC showed the reaction was completed.

The reaction mixture was diluted with H₂O (20 mL) and extracted with EA (20 mL). The organic layer was washed with saturated brine, dried over anhydrous magnesium sulphate and evaporated under reduced pressure. The residue was purified by SGC (eluent: PE:EA=4:1) to obtain 196 mg (yield 82.99%) of the target compound as white solid.

Compound No. 156: ¹H NMR: 10.59 (s, 1H), 8.50-8.39 (m, 3H), 7.96 (s, 1H), 7.79 (s, 1H), 7.80-7.77 (m, 2H), 7.74-7.68 (m, 1H), 7.57-7.47 (m, 2H), 7.29 (dd, J=7.7, 4.8 Hz, 1H), 3.81 (d, J=6.8 Hz, 2H), 1.12-1.00 (m, 1H), 0.48-0.38 (m, 2H), 0.15 (d, J=4.5 Hz, 2H).

In addition to the compounds described in the examples, compounds in Table 1 can be prepared according to the similar methods as described in examples 1-7. Hereinbelow, Table 3 shows the NMR data of some compounds prepared according to examples 1-7.

TABLE 3

| Compound | |
|---|---|
| No. | ¹H NMR (DMSO-d₆, δ: ppm) |
| 13 | 10.33 (s, 1H), 7.91 (s, 1H), 7.80-7.77 (m, 2H), 7.54 (s, 1H), 7.47-7.41 (m, 2H), 7.33 (t, J = 72 |
| | Hz, 1H), 7.32-7.21 (m, 5H), 3.78 (d, J = 8 Hz, 2H), 1.07-1.01 (m, 1H), 0.44-0.40 (m, 2H), |
| | 0.14-0.11 (m, 2H). |
| 14 | 10.29 (s, 1H), 7.91 (s, 1H), 7.74-7.66 (m, 3H), 7.57-7.46(m, 4H), 7.33-7.31 (m, 2H), 3.73 (s, |
| | 2H), 1.04-1.00 (m, 1H), 0.42 (d, J = 8.0 Hz, 2H), 0.13 (d, J = 16.0 Hz, 2H). |
| 27 | 10.29 (s, 1H), 7.90 (s, 1H), 7.74-7.50 (m, 7H), 7.33-7.31 (m, 2H), 3.74 (d, J = 16.0 Hz, 2H), |
| | 1.05-1.02 (m, 1H), 0.43 (d, J = 8.0 Hz, 2H), 0.14 (d, J = 8.0 Hz, 2H). |
| 28 | 10.33 (s, 1H), 7.91 (s, 1H), 7.80 (d, J = 12 Hz, 2H), 7.61 (d, J = 8 Hz, 2H), 7.54-7.47 (m, 5H), |
| | 7.30 (t, J = 72 Hz, 1H), 3.81 (d, J = 8 Hz, 2H), 1.08-1.02 (m, 1H), 0.46-0.41 (m, 2H), |
| | 0.16-0.15 (m,2H). |
| 43 | 10.33 (s, 1H), 7.91 (s, 1H), 7.80-7.77 (m, 2H), 7.54 (s, 1H), 7.50-7.44 (m, 2H), 7.37-7.34 (m, |
| | 2H), 7.32 (t, J = 72 Hz, 1H), 7.07 (t, J = 8 Hz, 2H), 3.78 (d, J = 4 Hz, 2H), 1.06-1.00 (m, 1H), |
| | 0.44-0.40 (m, 2H), 0.15-0.11 (m, 2H). |
| 44 | 10.31 (s, 1H), 7.90 (s, 1H), 7.68-7.48 (m, 3H), 7.33 (t, J = 72.0 Hz, 2H), 7.32 (brs, 6H), 3.69 |
| | (d, J = 16.8 Hz, 2H), 1.02 (brs, 1H), 0.41 (d, J = 7.7 Hz, 2H), 0.09 (s, 2H). |
| 55 | 7.92 (t, J = 7.2 Hz, 2H), 7.71 (d, J = 1.9 Hz, 1H), 7.59-7.35 (m, 2H), 7.23 (s, 2H), 7.18-7.04 |
| | (m, 2H), 6.48 (t, J = 73.1 Hz, 1H), 3.70 (s, 2H), 1.16 (s, 9H), 1.04 (d, J = 6.2 Hz, 1H), 0.41 (d, |
| | J = 8.0 Hz, 2H), 0.22-0.02 (m, 2H). |
| 61 | 10.32 (s, 1H), 7.90 (d, J = 1.9 Hz, 1H), 7.80 (d, J = 7.8 Hz, 2H), 7.69 (d, J = 7.1 Hz, 1H), 7.55 |
| | (d, J = 9.0 Hz, 4H), 7.33 (s, 1H), 7.32 (t, J = 72.8 Hz, 1H), 3.79 (s, 1H), 3.70(s, 1H), 3.17 (s, |
| | 3H), 1.03 (s, 1H), 0.43 (d, J = 8.1 Hz, 2H), 0.13 (s, 2H). |
| 77 | 10.29 (s, 1H), 7.90 (s, 1H), 7.62-7.49(m, 3H), 7.50-7.38 (m, 1H), 7.36-7.25 (m, 2H), 7.20-7.08 |
| | (m, 1H), 6.98 (t, J = 9.3 Hz, 1H), 3.88-3.76 (m, 1H), 3.65 (dd, J = 13.6, 7.0 Hz, 1H), |
| | 1.05-0.99 (m, 1H), 0.44 (d, J = 7.9 Hz, 2H), 0.21-0.06 (m, 2H). |
| 78 | 10.31 (s, 1H), 7.91 (s, 1H), 7.67-7.48 (m, 4H), 7.38-7.26 (m, 3H), 7.33 (t, J = 72 Hz, 1H), 3.89 |
| | (dd, J = 13.8, 7.1 Hz, 1H), 3.57 (dd, J = 13.9, 7.0 Hz, 1H), 1.07-0.98 (m, 1H), 0.46 (d, J = 8.1 |
| | Hz, 2H), 0.17 (d, J = 3.7 Hz, 2H). |
| 81 | 10.32 (s, 1H), 7.90 (s, 1H), 7.67 (t, J = 6.5 Hz, 1H), 7.64-7.47 (m, 1H), 7.32 (brs, 3H), 7.14 (s, |
| | 3H), 3.70 (d, J = 38.8 Hz, 2H), 1.02 (brs, 1H), 0.42 (d, J = 7.6 Hz, 2H), 0.11 (brs, 2H). |
| 83 | 10.33 (s, 1H), 7.90 (s, 1H), 7.68-7.47 (m, 4H), 7.35-7.27 (m, 1H), 7.28-7.07 (m, 3H), 3.8 (dd, |
| | J = 13.9, 7.1 Hz, 1H), 3.62 (dd, J = 13.8, 7.2 Hz, 1H), 1.08-0.96 (m, 1H), 0.44 (d, J = 7.9 Hz, |
| | 2H), 0.15 (s, 2H). |
| 85 | 10.34 (s, 1H), 7.91 (s, 1H), 7.73 (t, J = 7.3 Hz, 1H), 7.61 (s, 1H), 7.52 (s, 1H), 7.35 (t, J = 8.0 |
| | Hz, 1H), 7.31 (t, J = 72.0 Hz, 1H), 7.23 (s, 2H), 7.00 (s, 2H), 3.69 (s, 2H), 1.00 (brs, 1H), 0.42 |
| | (d, J = 7.2 Hz, 2H), 0.09 (s, 2H). |
| 98 | 10.29 (s, 1H), 7.90 (s, 1H), 7.81 (d, J = 17.9 Hz, 1H), 7.72 (s, 1H), 7.61-7.44 (m, 5H), 7.34 |
| | (dd, J = 16.9, 8.0 Hz, 2H), 3.87-3.70 (m, 2H), 1.04 (brs, 1H), 0.51-0.42 (m, 2H), 0.21 (d, J = |
| | 33.7 Hz, 2H). |
| 100 | 10.63 (s, 1H), 8.68-8.32 (m, 3H), 7.95 (s, 1H), 7.85-7.66 (m, 2H), 7.60 (s, 1H), 7.45-7.24 (m, |
| | 2H), 3.74 (br s, 2H), 1.03 (br s, 1H), 0.42 (d, J = 7.0 Hz, 2H), 0.10 (br s, 2H). |
| 101 | 10.60 (s, 1H), 8.56-8.37 (m, 3H), 7.96 (s, 1H), 7.73 (s, 1H), 7.60 (s, 1H), 7.34 (t, J = 7.5 Hz, |
| | 1H), 7.30-7.12 (m, 2H), 3.74 (d, J = 6.3 Hz, 2H), 1.03 (br s, 1H), 0.43 (d, J = 6.9 Hz, 2H), |
| | 0.13 (d, J = 13.2 Hz, 2H). |
| 105 | 10.63 (s, 1H), 8.42 (s, 1H), 8.30 (s, 1H), 7.95 (s, 1H), 7.79 (t, J = 7.0 Hz, 2H), 7.63 (s, 1H), |
| | 7.49-7.36 (m, 2H), 3.75 (d, J = 40.6 Hz, 2H), 1.03 (br s, 1H), 0.42 (d, J = 6.3 Hz, 2H), 0.12 (d, |
| | J = 28.2 Hz, 2H). |
| 108 | 10.56 (s, 1H), 8.42 (s, 1H), 8.21 (d, J = 8.0 Hz, 1H), 7.95 (s, 1H), 7.88 (d, J = 7.2 Hz, 1H), |
| | 7.82-7.79 (m, 2H), 7,65-7.63 (m, 1H), 7.39-7.36 (m, 1H), 3.86 (br s, 2H), 1.05 (br s, 1H), |
| | 0.47 (d, J = 7.6 Hz, 2H), 0.20-0.18 (m, 2H). |
| 109 | 10.59 (s, 1H), 8.42 (s, 1H), 7.96 (s, 1H), 7.77-7.68(m, 2H), 7.59 (t, J = 6.4 Hz, 1H), 7.32 (t, J = |
| | 7.6 Hz, 1H), 7.17 (d, J = 8.0 Hz, 1H), 3.84 (br s, 1H), 3.65 (br s, 1H), 1.02 (br s, 1H), 0.45 |
| | (d, J = 8.0 Hz, 2H), 0.16 (br s, 2H). |
| 110 | 10.63 (s, 1H), 8.42 (s, 1H), 8.30 (d, J = 4.8 Hz, 1H), 7.96 (s, 1H), 7.79 (t, J = 7.2 Hz, 1H), |
| | 7.62-7.61(m, 1H), 7.42-7.36(m, 2H), 7.25 (d, J = 4.0 Hz, 1H), 3.79-3.67(m, 2H), |
| | 1.05-1.00(m, 1H), 0.43 (d, J = 7.6 Hz, 2H), 0.13 (d, J = 22.8 Hz, 2H). |
| 111 | 10.51 (s, 1H), 8.31 (s, 1H), 8.03 (d, J = 5.2 Hz, 1H), 7.85 (s, 1H), 7.67 (t, J = 7.6 Hz, 1H), |
| | 7.51(t, J = 6.8 Hz, 1H), 7.26(t, J = 8.0 Hz, 1H) 7.10 (s, 1H), 6.99(s, 1H), 3.70-3.55(m, 2H), |
| | 0.92(brs, 1H), 0.33 (d, J = 8.0 Hz, 2H), 0.03 (d, J = 22.0 Hz, 2H). |
| 112 | 10.36 (s, 1H), 8.47 (s, 2H), 7.90 (s, 1H), 7.77-7.66 (m, 2H), 7.64-7.48 (m, 3H), 7.32 (m, 2H), |
| | 3.86-3.60 (m, 2H), 1.02 (brs, 1H), 0.42 (d, J = 7.7 Hz, 2H), 0.20-0.03 (m, 2H). |
| 113 | 10.33 (s, 1H), 8.47 (d, J = 3.9 Hz, 2H), 7.91 (s, 1H), 7.69 (t, J = 6.0 Hz, 1H), 7.56 (d, J = 12.9 |
| | Hz, 2H), 7.36-7.18 (m, 4H), 3.90-3.59 (m, 2H), 1.03 (brs, 1H), 0.43 (d, J = 7.7 Hz, 2H), 0.13 |
| | (s, 2H). |
| 117 | 10.37 (s, 1H), 8.30(s, 1H), 7.91(s, 1H), 7.80-7.73(m, 2H), 7.65-7.53(m, 2H), 7.45-7.35(m, |
| | 2H), 7.32 (t, J = 72.0 Hz, 1H), 3.73 (d, J = 8.0 Hz, 2H), 1.04-1.01(m, 1H), 0.42 (d, J = 8.0 Hz, |
| | 2H), 0.13(d, J= 12.0 Hz, 2H). |
| 119 | 10.36 (s, 1H), 8.31 (d, J = 4.8 Hz, 1H), 7.91 (s, 1H), 7.77 (t, J = 7.8 Hz, 1H), 7.66-7.49 (m, |
| | 2H), 7.48-7.31 (m, 2H), 7.32 (t, J = 72.0 Hz, 1H), 7.25 (d, J = 4.8 Hz, 1H), 3.71 (q, J = 13.9, |
| | 10.9 Hz, 2H), 1.03 (brs, 1H), 0.44 (d, J = 7.8 Hz, 2H), 0.14 (dd, J = 11.4, 4.2 Hz, 2H). |
| 120 | 10.22 (s, 1H), 8.01 (d, J = 4.8 Hz, 1H), 7.78 (s, 1H), 7.62 (t, J = 7.2 Hz, 1H), 7.50-7.44 (m, |
| | 1H), 7.41 (s, 1H), 7.24-7.20 (m, 1H), 7.32 (t, J = 72.0 Hz, 1H), 7.08 (d, J = 4.8 Hz, 1H), |
| | 6.97(br s, 1H), 3.67-3.55 (m, 2H), 1.29 (brs, 1H), 0.31 (d, J = 8.4 Hz, 2H), 0.06-0.04 (m, 2H). |
| 121 | 10.56 (d, J = 28.9 Hz, 1H), 8.52-8.35 (m, 3H), 7.95 (s, 1H), 7.79 (d, J = 24.4 Hz, 1H), 7.65 (d, |
| | J = 30.2 Hz, 2H, 7.37 (t, J = 7.6 Hz, 1H), 7.26 (s, 1H), 4.06 (br s, 1H), 1.41 (br s, 1H), 1.24 (s, |
| | 3H), 0.55 (d, J = 47.9 Hz, 2H), 0.35 (d, J = 43.8 Hz, 2H). |
| 122 | 10.56 (d, J = 29.4 Hz, 1H), 8.44 (d, J = 4.4 Hz, 2H), 8.42 (s, 1H), 7.96 (s, 1H), 7.75 (dt, J = |
| | 20.3, 7.2 Hz, 1H), 7.61 (s, 1H), 7.35 (t, J = 7.7 Hz, 1H), 7.22 (s, 2H), 4.13-3.99 (m, 1H), 1.40 |
| | (d, J = 6.0 Hz, 1H), 1.24 (s, 3H), 0.55 (d, J = 41.6 Hz, 2H), 0.36 (d, J = 47.0 Hz, 2H). |
| 123 | 10.57 (d, J = 26.5 Hz, 1H), 8.41 (s, 1H), 8.12 (s, 1H), 7.95 (s, 1H), 7.93-7.69 (m, 2H), 7.64 (s, |
| | 1H), 7.41 (t, J = 7.7 Hz, 1H), 7.07 (d, J = 6.0 Hz, 1H), 4.06 (br s, 1H), 1.42 (br s, 1H), 1.24 (s, |
| | 3H), 0.55 (d, J = 51.6 Hz, 2H), 0.35 (d, J = 37.2 Hz, 2H). |
| 144 | 10.27 (s, 1H), 8.64 (d, J = 2.2 Hz, 1H), 8.30-8.22 (m, 1H), 7.99-7.84 (m, 2H), 7.63-7.51 (m, |
| | 3H), 7.31 (t, J = 72.8 Hz, 1H), 7.25 (t, J = 7.8 Hz, 1H), 3.91 (dd, J = 14.0, 7.0 Hz, 1H), 3.65 |
| | (dd, J = 14.1, 7.3 Hz, 1H), 1.06 (s, 1H), 0.45 (d, J = 7.8 Hz, 2H), 0.17 (d, J = 5.0 Hz, 2H). |
| 145 | 10.26 (s, 1H), 8.22 (s, 1H), 7.91 (d, J = 2.0 Hz, 1H), 7.85-7.70 (m, 2H), 7.54 (s, 2H), 7.51 (s, |
| | 1H), 7.33 (t, J = 72 Hz, 1H), 7.24 (t, 7=8.1 Hz, 1H), 3.86 (d, J = 8.4 Hz, 1H), 3.59 (d, J = 6.7 |
| | Hz, 1H), 1.12-0.98 (m, 1H), 0.43 (d, J = 7.8 Hz, 2H), 0.15 (d, J = 4.8 Hz, 2H). |
| 146 | 10.28 (s, 1H), 8.28 (s, 1H), 7.96 (d, J = 8.5 Hz, 1H), 7.91 (s, 1H), 7.70 (d, J = 8.5 Hz, 1H), |
| | 7.53 (d, J = 10.6 Hz, 2H), 7.51 (m, 1H), 7.33 (t, J = 72.8 Hz, 1H), 7.25 (t, J = 7.9 Hz, 1H), |
| | 3.88 (dd, J = 14.2, 6.9 Hz, 1H), 3.61 (dd, J = 14.1, 7.3 Hz, 1H), 1.05 (s, 1H), 0.44 (d, J = 7.7 |
| | Hz, 2H), 0.16 (d, J = 5.1 Hz, 2H). |
| 147 | 10.29 (s, 1H), 8.35 (s, 1H), 8.14-8.04 (m, 1H), 7.91 (d, J = 2.0 Hz, 1H), 7.63 (d, J = 8.4 Hz, |
| | 1H), 7.53 (d, J = 11.0 Hz, 3H), 7.33 (t, J = 72.0 Hz, 1H), 7.25 (t, J = 7.6 Hz, 1H), 3.88 (dd, J = |
| | 14.1, 7.0 Hz, 1H), 3.60 (dd, J = 14.4, 7.2 Hz, 1H), 1.10-1.00 (m, 1H), 0.43 (d, J = 7.8 Hz, 2H), |
| | 0.15 (d, J = 4.7 Hz, 2H). |
| 148 | 10.27 (s, 1H), 9.02 (d, J = 2.6 Hz, 1H), 8.60 (dd, J = 8.6, 2.6 Hz, 1H), 7.97-7.88 (m, 2H), |
| | 7.63-7.52 (m, 3H), 7.32 (t, J = 72.4 Hz, 1H), 7.26 (t, J = 7.8 Hz, 1H), 3.91 (dd, J = 14.0, 7.0 |
| | Hz, 1H), 3.66 (dd, J = 13.9, 7.2 Hz, 1H), 1.06 (s, 1H), 0.46 (h, J = 4.3 Hz, 2H), 0.22-0.14 (m, |
| | 2H). |
| 149 | 10.28 (s, 1H), 8.71 (d, J = 2.0 Hz, 1H), 8.35 (dd, J = 8.2, 2.1 Hz, 1H), 7.96-7.88 (m, 1H), 7.84 |
| | (d, J = 8.2 Hz, 1H), 7.55 (d, J = 8.2 Hz, 3H), 7.33 (t, J = 72.8 Hz, 1H), 7.26 (t, J = 7.8 Hz, |
| | 1H), 3.89 (dd, J = 14.0, 7.0 Hz, 1H), 3.65 (dd, J = 14.1, 7.2 Hz, 1H), 1.03 (dt, J = 13.3, 7.2 Hz, |
| | 1H), 0.44 (d, J = 7.9 Hz, 2H), 0.17 (d, J = 5.0 Hz, 2H). |
| 150 | 10.50 (d, J = 12.5 Hz, 1H), 8.63 (s, 1H), 8.41 (s, 1H), 8.26 (d, J = 7.4 Hz, 1H), 7.95 (s, 1H), |
| | 7.88 (d, J = 8.1 Hz, 1H), 7.61 (dt, J = 13.2, 6.7 Hz, 2H), 7.30 (t, J = 7.1 Hz, 1H), 3.82 (d, J = |
| | 25.2 Hz, 2H), 1.06 (s, 1H), 0.45 (d, J = 7.8 Hz, 2H), 0.19 (s, 2H). |
| 152 | 10.55 (s, 1H), 8.42 (s, 1H), 8.27 (s, 1H), 7.96 (s, 2H), 7.71 (d, J = 8.4 Hz, 1H), 7.61-7.52 (m, |
| | 2H), 7.28 (t, J = 7.6 Hz, 1H), 3.82 (s, 1H), 3.69 (s, 1H), 1.05 (s, 1H), 0.44 (d, J = 7.5 Hz, 2H), |
| | 0.16 (s, 2H). |
| 153 | 10.54 (s, 1H), 8.70 (s, 1H), 8.42 (s, 1H), 8.36 (d, J = 9.7 Hz, 1H), 7.96 (s, 1H), 7.84 (d, J = 8.1 |
| | Hz, 1H), 7.59 (dt, J = 12.4, 7.0 Hz, 2H), 7.29 (t, J = 7.7 Hz, 1H), 3.78 (d, J = 28.6 Hz, 2H), |
| | 1.04 (s, 1H), 0.45 (d, J = 8.0 Hz, 2H), 0.17 (s, 2H). |
| 154 | 10.53 (s, 1H), 9.04-8.98 (m, 1H), 8.61 (dd, J = 8.6, 2.4 Hz, 1H), 8.42 (s, 1H), 7.95 (s, 2H), |
| | 7.61 (dt, J = 22.0, 6.6 Hz, 2H), 7.29 (t, J = 7.5 Hz, 1H), 3.80 (d, J = 43.6 Hz, 2H), 1.05 (s, |
| | 1H), 0.46 (d, J = 7.9 Hz, 2H), 0.18 (s, 2H). |
| 157 | 10.58 (s, 1H), 8.46 (s, 1H), 8.43 (s, 1H), 7.96 (s, 1H), 7.79 (d, J = 8.1 Hz, 2H), 7.53 (dt, J = |
| | 15.1, 7.5 Hz, 2H), 7.25 (s, 2H), 3.78 (s, 2H), 1.04 (dd, J = 12.7, 6.1 Hz, 1H), 0.44 (d, J = 7.0 |
| | Hz, 2H), 0.15 (s, 2H). |
| 161 | 10.58 (s, 1H), 8.43 (s, 1H), 8.13 (s, 1H), 7.97 (s, 1H), 7.90 (t, J = 8.7 Hz, 1H), 7.81 (d, J = 7.4 |
| | Hz, 1H), 7.75 (s, 1H), 7.55 (dt, J = 15.4, 8.0 Hz, 2H), 7.10 (d, J = 10.7 Hz, 1H), 3.82 (d, J = |
| | 7.0 Hz, 2H), 1.10-0.97 (m, 1H), 0.49-0.40 (m, 2H), 0.15 (d, J = 4.5 Hz, 2H). |
| 164 | 10.34 (s, 1H), 8.44 (d, J = 2.7 Hz, 2H), 7.91 (s, 1H), 7.81 (d, J = 12.9 Hz, 2H), 7.71 (d, J = 7.8 |
| | Hz, 1H), 7.54 (s, 1H), 7.52-7.45 (m, 2H), 7.32 (t, J = 76.0 Hz, 1H), 7.34-7.25 (m, 1H), 3.80 |
| | (d, 7= 7.0 Hz, 2H), 1.08-1.03 (m, 1H), 0.43 (d, J = 18.1 Hz, 2H), 0.14 (d, J = 4.6 Hz, 2H). |
| 165 | 10.34 (s, 1H), 8.46 (s, 2H), 7.91 (s, 1H), 7.82 (d, J = 9.3 Hz, 2H), 7.57-7.45 (m, 3H), 7.31 (t, |
| | J = 72 Hz, 1H), 7.25 (s, 2H), 3.78 (s, 2H), 1.02 (brs, 1H), 0.43 (d, J = 7.1 Hz, 2H), 0.14 (s, 2H). |
| 168 | 10.34 (s, 1H), 8.28 (s, 1H), 7.91 (s, 1H), 7.83 (d, J = 9.0 Hz, 2H), 7.77 (d, J = 7.9 Hz, 1H), |
| | 7.54 (d, J = 6.7 Hz, 2H), 7.50 (d, J = 6.8 Hz, 1H), 7.42 (d, J = 8.2 Hz, 1H), 7.31 (t, J = 72.0 |
| | Hz, 1H), 3.80 (d, J = 6.9 Hz, 2H), 1.11-0.97 (m, 1H), 0.48-0.39 (m, 2H), 0.14 (d, J = 4.5 Hz, |
| | 2H). |
| 169 | 10.34 (s, 1H), 8.14 (s, 1H), 7.91 (s, 2H), 7.82 (d, J = 8.9 Hz, 2H), 7.53 (d, J = 7.9 Hz, 2H), |
| | 7.50 (d, J = 6.8 Hz, 1H), 7.31 (t, J = 72.0 Hz, 1H), 7.09 (dd, J = 8.5, 2.2 Hz, 1H), 3.81 (d, J = |
| | 7.0 Hz, 2H), 1.04 (brs, 1H), 0.43 (d, J = 9.5 Hz, 2H), 0.14 (d, J = 4.8 Hz, 2H). |
| 170 | 10.35 (s, 1H), 8.28 (br s, 1H), 7.92 (br s, 1H), 7.90-7.81 (m, 2H), 7.60-7.49 (m, 4H), 7.43 (s, |
| | 1H), 7.32 (t, J = 72.0 Hz, 1H), 7.28 (s, 1H), 3.77 (s, 2H), 1.09-0.98 (m, 1H), 0.44 (d, J = 7.3 |
| | Hz, 2H), 0.15 (s, 2H). |
| 173 | 10.34 (s, 1H), 8.32 (dd, J = 4.7, 1.3 Hz, 1H), 7.91 (td, J = 6.8, 5.7, 1.6 Hz, 2H), 7.54 (td, J = |
| | 9.8, 9.2, 3.3 Hz, 2H), 7.50-7.43 (m, 1H), 7.34 (t, J = 72.7 Hz, 1H), 7.33-7.30 (m, 1H), 7.21 (d, |
| | J = 7.8 Hz, 1H), 4.10 (dd, J = 14.2, 7.1 Hz, 1H), 3.45 (s, 1H), 1.02 (tq, J = 12.4, 7.4, 6.1 Hz, |
| | 1H), 0.52-0.42 (m, 2H), 0.21 (d, J = 19.6 Hz, 2H). |
| 174 | 10.62 (s, 1H), 8.42 (s, 1H), 8.32 (dd, J = 4.7, 1.2 Hz, 1H), 7.96 (s, 1H), 7.88 (dd, J = 8.2, 1.2 |
| | Hz, 1H), 7.56 (d, J = 6.3 Hz, 1H), 7.51-7.46 (m, 1H), 7.37-7.28 (m, 1H), 7.22 (t, J = 7.8 Hz, |
| | 1H), 4.06 (d, J = 15.6 Hz, 1H), 3.48 (d, J = 13.3 Hz, 1H), 1.03 (d, J = 6.2 Hz, 1H), 0.48 (d, J = |
| | 8.0 Hz, 2H), 0.21 (d, J = 14.1 Hz, 2H). |
| 175 | 10.64 (s, 1H), 8.43 (s, 2H), 8.25 (d, J = 2.0 Hz, 1H), 7.96 (s, 1H), 7.62-7.48 (m, 2H), 7.27 (t, |
| | J = 7.8 Hz, 1H), 4.00 (s, 1H), 3.57 (s, 1H), 1.02 (s, 1H), 0.48 (d, J = 7.9 Hz, 2H), 0.21 (s, 2H). |
| 176 | 10.29 (s, 1H), 8.34-8.21 (m, 1H), 7.96 (dd, J = 8.3, 2.4 Hz, 1H), 7.91 (d, J = 2.0 Hz, 1H), 7.71 |
| | (d, J = 8.5 Hz, 1H), 7.55 (d, J = 9.4 Hz, 1H), 7.51 (d, J = 3.4 Hz, 1H), 7.33 (t, J = 73.6 Hz, |
| | 1H), 7.29-7.20 (m, 1H), 3.88 (dd, J = 13.9, 6.5 Hz, 1H), 3.61 (dd, J = 14.5, 7.3 Hz, 1H), 1.04 |
| | (q, J = 6.5, 5.3 Hz, 1H), 0.44 (d, J = 7.8 Hz, 2H), 0.16 (d, J = 5.0 Hz, 2H). |
| 177 | 10.32 (s, 1H), 7.90 (d, J = 1.9 Hz, 1H), 7.80 (d, J = 7.8 Hz, 2H), 7.69 (d, J =7.1 Hz, 1H), 7.55 |
| | (d, J = 9.0 Hz, 4H), 7.33 (s, 1H), 7.32 (t, J = 72.8 Hz, 1H), 3.79 (s, 1H), 3.70(s, 1H), 3.17 (s, |
| | 3H), 1.03 (s, 1H), 0.43 (d, J = 8.1 Hz, 2H), 0.13 (s, 2H). |
| 178 | 8.08 (d, J = 13.6 Hz, 1H), 8.02 (s, 1H), 7.88-7.75 (m, 1H), 7.68 (dd, J = 7.7, 2.1 Hz, 1H), 7.50 |
| | (d, J = 1.9 Hz, 1H), 7.48-7.41 (m, 1H), 7.22 (t, J = 7.9 Hz, 1H), 6.83 (dd, J = 8.2, 2.9 Hz, |
| | 1H), 6.58 (t, J = 73.0 Hz, 1H), 3.90 (dd, J = 14.0, 7.3 Hz, 1H), 3.73 (dd, J = 13.3, 7.1 Hz, 2H), |
| | 1.13 (dt, J = 7.6, 4.2 Hz, 1H), 0.52 (dd, J = 8.0, 3.3 Hz, 2H), 0.22 (d, J = 5.0 Hz, 2H). |
| 179 | 10.55 (s, 1H), 8.42 (s, 1H), 8.01-7.94 (m, 2H), 7.58 (dd, J = 12.7, 6.7 Hz, 3H), 7.29 (t, J = 7.5 |
| | Hz, 1H), 7.13 (d, J = 7.7 Hz, 1H), 3.80 (s, 1H), 3.65 (dd, J = 12.8, 5.8 Hz, 1H), 1.04 (s, 1H), |
| | 0.44 (d, J = 7.4 Hz, 2H), 0.16 (s, 2H). |
| 180 | 10.28 (s, 1H), 7.91 (s, 1H), 7.70-7.65 (m, 1H), 7.63-7.57 (m, 1H), 7.54 (s, 1H), 7.50 (s, 0H), |
| | 7.43 (s, 1H), 7.32 (s, 2H), 7.20 (s, 1H), 7.14 (s, 0H), 3.72 (d, J =J = 22.3 Hz, 2H), 2.38 (s, 3H), |
| | 1.02 (s, 1H), 0.43 (d, J = 6.6 Hz, 2H), 0.14 (s, 2H). |
| 181 | 10.30 (s, 1H), 7.91 (s, 1H), 7.77 (d, J = 8.7 Hz, 1H), 7.68-7.52 (m, 5H), 7.50 (s, 0H), 7.31 (d, |
| | J = 4.9 Hz, 1H), 7.14 (s, 0H), 3.82 (dd, J = 13.8, 7.3 Hz, 1H), 3.70 (dd, J = 14.0, 7.0 Hz, 1H), |
| | 1.06-0.97 (m, 1H), 0.45 (d, J = 8.0 Hz, 2H), 0.16 (dd, J = 8.6, 4.7 Hz, 2H). |
| 182 | 10.28 (s, 1H), 7.93-7.87 (m, 2H), 7.65 (d, J = 10.4 Hz, 2H), 7.55 (d, J = 6.9 Hz, 2H), 7.51 (s, |
| | 0H), 7.47 (d, J = 7.9 Hz, 1H), 7.33 (s, 0H), 7.28 (d, J = 9.4 Hz, 2H), 7.14 (s, 0H), 3.90-3.81 |
| | (m, 1H), 3.70-3.60 (m, 1H), 2.34 (s, 3H), 1.07-1.00 (m, 1H), 0.46 (d, J = 8.0 Hz, 2H), 0.18 (s, |
| | 2H). |

Other compounds represented by general formula I of this invention can also be prepared according to the methods described above.

### FORMULATION EXAMPLES

### Formulation Example 1

In the embodiment, compound 1 of the invention is used as a representative compound to prepare a formulation. The details are as follows:

30 parts (by weight, the other ingredients of this example and formulation examples below are all by weight) of compound 1, 15 parts of polyoxyethylene styrylphenyl ether, 10 parts of phosphite and 45 parts of xylene are evenly mixed to obtain the 30% emulsion of compound 1.

### Formulation Example 2

In the present embodiment, compound 27 of the invention is used as a representative compound to prepare a formulation. The details are as follows:

20 parts of compound 27, 2 parts of sodium dodecyl sulfate, 2 parts of dialkylsulphonate succinate, 1 part of sodium salt of 0-naphthalenesulfonate formaldehyde condensate and 75 parts of diatomite were evenly stirred and mixed to obtain 20% wettable powder of compound 27.

### Formulation Example 3

In the embodiment, compound 43 of the invention is used as a representative compound to prepare a formulation. The details are as follows:

30 parts of compound 43 of the invention, 10 parts of ethylene glycol, 6 parts of nonylphenol polyethylene glycol ether, 10 parts of sodium lignosulfonate, 10 parts of carboxymethyl cellulose and 1 part of silicone oil aqueous solution, 33 parts of water were evenly stirred and mixed to obtain 30% suspending agent of compound 43.

### Examples for Bioactivity Tests

Various kinds of pests were tested with the compounds of this invention. Unless otherwise specified, the preparation method of samples and definition of the mortality rate of the insects in the embodiments and this invention are as follows: the preparation method of samples is to weigh 10 mg of the compound and dissolve it in 1 mL DMF to prepare 10,000 ppm mother liquid, which is diluted to necessary concentration by 0.05% Tween-80 water, respectively. The mortality rate is the mortality rate of pests under the test concentration, whose calculating formula is: mortality rate (%)=Number of dead pests/total pests*100

### Example 1 of Biological Test: Insecticidal Activity Test Against Mythimna separata

The leaf dip method was used to assay the insecticidal activity. Cut above ground part of fresh maize seedlings (about 10 cm). Dip the maize seedlings into the solution prepared with compound of this invention for 10 seconds and dry them in a cool environment. Then cut the dry maize seedlings into 3-5 cm leaf sections and put 3 leaf sections into each petri dish. Put ten of 3th-instar larvae of Mythimna separatas into each dish, which was repeated by 3 times. Then the dishes were placed in an illumination incubator and incubated with 14 hL: 10 hD illumination at 25° C. Symptoms were investigated on the 1st, 2nd and 3rd day after treatment, and the mortality was calculated.

The insecticidal activity of compounds 55, 144, 145, 146, 147, 148, 149, 150, 152, 153, 154, 173, 174, 175, 176, 177, 178 and 179 of this invention is ≥90% (mortality of Mythimna separate) at 1 ppm on the 3rd day after treatment.

The insecticidal activity of compounds 105, 110 and 117 of this invention is ≥90% (mortality of Mythimna separate) at 0.1 ppm on the 3rd day after treatment.

The insecticidal activity of compounds 1, 31, 106, 111, 118 and 120 of this invention is ≥90% (mortality of Mythimna separate) at 0.04 ppm on the 3rd day after treatment.

According to the above method, compound 31 and KC1 were selected and parallelly tested against Mythimna separate to compare their insecticidal activity. The results are shown in Table 4.

TABLE 4
Mortality of compound 31 and KC1 against Mythimna separate

Mortality (%)

| Compound | concentration | 1 d | 2 d | 3 d |
|---|---|---|---|---|
| 31 | 0.01 ppm | 13.33 | 63.33 | 70.00 |
| KC1 | 0.01 ppm | 6.67 | 16.67 | 20.00 |

### Example 2 of Biological Test: Insecticidal Activity Test Against Spodoptera litura

The leaf dip method was used to assay the insecticidal activity. Healthy and pesticide-untreated cabbage leaves was selected to prepare 1 cm of leaf discs by diameter. Dip the leaf discs into the solution prepared with compound of this invention for 10 seconds and dry them in a cool 30 environment. Then place them in 24-well plate with 3 discs per pore. Put 10 of Spodoptera litura into each pore, which was repeated by 3 times. The 24-well plate was placed in an illumination incubator and incubated with 14 hL: 10 hD illumination at 25° C. The dead number of Spodoptera litura was investigated on the 3rd day after treatment, and the mortality was calculated.

The insecticidal activity of some compounds of this invention against Spodoptera litura is as follows:

The insecticidal activity of compounds 1, 14, 27, 31, 44, 77, 83, 85, 106, 118, 119, 120 is ≥90% (mortality of Spodoptera litura) at 0.4 ppm on the 3rd day after treatment.

According to the above method, compound 118 and KC4 were selected and parallelly tested against Spodoptera litura to compare the insecticidal activity. The results are shown in Table 5.

TABLE 5
Mortality of compound 118 and KC1 against Spodoptera litura

Mortality (%, 3 d)

| compound | 0.1 ppm | 0.04 ppm |
|---|---|---|
| 118 | 95.83 | 66.67 |
| KC4 | 75.0 | 8.83 |

### Example 3 of Biological Test: Insecticidal Activity Test Against Chilo suppressalis

The rice was cultivated in a plastic pot with a diameter of 9 cm and a height of 10 cm. When the rice grew to 25 cm, the aerial part of robust and consistent rice seedlings was selectively cut. Their leaves were removed and their stems of about 8 cm were kept for use. Pour the solution prepared with compound of this invention into the Petri dish (about 40 mL) and dip the rice stems into the solution for 10 seconds. Take rice stems out and dry them in a cool environment. Put a wet cotton ball at the bottom of finger-like glass tube and 5 rice stems in each tube. Put 10 of 3rd-instar larvae of Chilo suppressa into each tube, which was repeated by 3 times. Seal the tubes with black cotton cloth and tighten them with rubber band. The tubes were placed in a illumination incubator at 28° C. and incubated in the dark (incubated without light). The dead number of Chilo suppressalis was investigated 3 days after treatment. The mortality was calculated.

The insecticidal activity of some compounds of this invention against Chilo suppressalis is as follows:

The insecticidal activity of compounds 110 and 124 is ≥90% (mortality of Chilo suppressalis) at 2 ppm on the 3rd day after treatment.

The insecticidal activity of compounds 1, 14, 27, 31, 44, 85, 106, 118 and 119 is ≥90% (mortality of Chilo suppressalis) at 1 ppm on the 3rd day after treatment.

According to the above method, compounds 1, 31 and KC3 were selected and parallelly tested against Chilo suppressalis. The results are shown in Table 6.

TABLE 6
Mortality of compounds 1,31 and KC3 against Chilo suppressalis

| | Mortality (%, 3 d) | | |
|---|---|---|---|
| compounds | 2 ppm | 1 ppm | 0.5 ppm |
| 1 | 100 | 96.67 | 76.67 |
| 31 | 100 | 100 | 83.33 |
| KC3 | 96.67 | 80.00 | 20.00 |

### Example 4 of Biological Test: Insecticidal Activity Test Against Aphis craccivora

Cut a single leaf of broad bean with stem and insert it into a glass jar filled with water (capacity of 20 ml). Five adult Aphis craccivoras were seeded onto each leaf, and covered with plastic cups with holes. The adult aphids were removed after 24 hours. Before the experiment, the base number was investigated and the single leaf with more than 15 nymphs aphids was selected for the experiment. Dip leaf of broad bean with nymphs aphids into the solution of test compound 10s, take out and dry them in a cool environment, 3 parallel repeats. Place the glass jars on the shelf of observation room and covered with plastic cups with holes. 20-25° C. with 14 hL: 10 hD illumination. The number of Aphis craccivora death and alive was investigated on the 3rd day after treatment, and the mortality was calculated.

The insecticidal activity of compounds 14, 27, 31, 44, 83, 101, 110, 111, 113, 118 and 120 is ≥90% (mortality of Aphis craccivora) at 40 ppm on the 3rd day after treatment.

According to the above method, compounds 101 and KC4 were selected and parallelly tested against Aphis craccivora to compare the insecticidal activity. The results are shown in Table 7.

TABLE 7
Mortality of compounds 101 and KC4 against Aphis craccivora

| | Mortality (%, 3 d) | | |
|---|---|---|---|
| compound | 40 ppm | 10 ppm | 1 ppm |
| 101 | 100.00 | 83.26 | 39.11 |
| KC4 | 65.23 | 47.15 | 0 |

### Example 5 of Biological Test: Insecticidal Activity Test Against Spodoptera frugiperda

The leaf dip method was used to assay the insecticidal activity. Cut above ground part of fresh maize seedlings (about 10 cm). Dip the maize seedlings into the solution prepared with compound of this invention for 10 seconds and dry them in a cool environment. Then cut the dry maize seedlings into 3-5 cm leaf sections and put 3 leaf sections into each petri dish. Put ten of 3th-instar larvae of Spodoptera frugiperda into each dish, which was repeated by 3 times. Then the dishes were placed in an illumination incubator and incubated with 14 hL: 10 hD illumination at 25° C. Symptoms were investigated on the 1st, 2nd and 3rd day after treatment, and the mortality was calculated.

The insecticidal activity of compounds 1, 14, 27, 31, 44, 77, 81, 83, 85, 105, 106, 111, 118, 119, 120 and 181 is ≥90% (mortality of Spodoptera frugiperda) at 1 ppm on the 3rd day after treatment.

According to the above method, some compounds of this invention, KC2 and KC3 were selected and parallelly tested against Spodoptera frugiperda to compare the insecticidal activity. The results are shown in Table 8. PGP-2 T

TABLE 8
Mortality of compounds of the invention, KC2
and KC3 against Spodoptera frugiperda

Mortality (%)

| compound | concentration | 1 d | 2 d | 3 d |
|---|---|---|---|---|
| 14 | 0.1 ppm | 29.17 | 79.17 | 95.83 |
| 44 | 0.1 ppm | 37.50 | 62.50 | 91.67 |
| 31 | 0.1 ppm | 100 | 100 | 100 |
| 106 | 0.1 ppm | 41.67 | 62.50 | 95.83 |
| 118 | 0.1 ppm | 29.17 | 66.67 | 91.67 |
| 119 | 0.1 ppm | 54.17 | 79.17 | 91.67 |
| 120 | 0.1 ppm | 58.33 | 100 | 100 |
| KC2 | 0.1 ppm | 0 | 8.33 | 20.83 |
| KC3 | 0.1 ppm | 0 | 0 | 4.17 |

The applicant states that the amide compounds of this invention, the preparation methods and applications thereof can be illustrated by the above examples, but this invention is not limited thereto, i.e., which does not mean that the implementation of this invention must rely on the above examples. Those skilled in the art should understand that any improvement to this invention, equivalent replacement of the raw materials for preparing the compounds of this invention, addition of auxiliary ingredients, selection of specific methods, etc., all fall within the scope of protection and disclosure of this invention.

1. An amide compound of formula I: 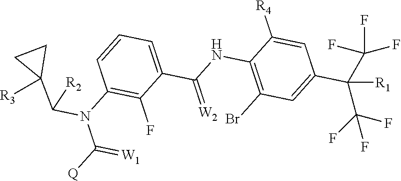
wherein,
Q is independently Q1, Q2, Q3 or Q4: 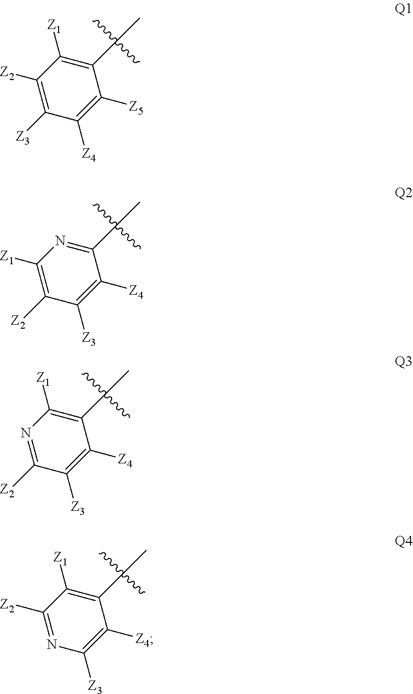
Z₁, Z₂, Z₃, Z₄, and Z₅ are independently of each other H, F, Cl, Br, I, CN, NO₂, C₁-C₆ alkyl, C₃-C₈ cycloalkyl, C₁-C₆ haloalkyl, C₃-C₈ halocycloalkyl, C₁-C₆ alkoxyl, C₁-C₆ haloalkoxyl, C₁-C₆ alkylsulfinyl, C₁-C₆ haloalkylsulfinyl, C₁-C₆ alkylsulfonyl, or C₁-C₆ haloalkylsulfonyl;
R₁ is H or F;
R₂ is H, C₁-C₆ alkyl, C₁-C₆ haloalkyl, C₃-C₈ cycloalkyl or C₃-C₈ halocycloalkyl;
R₃ is H or halogen;
R₄ is —OCF₂H or —CF₃, in a case when Q is Q1, R₄ is —OCF₂H; and
W₁ and W₂ are independently of each other O or S.

2. The amide compound according to claim 1, wherein, Z₁, Z₂, Z₃, Z₄, and Z₅ are independently of each other H, F, Cl, Br, I, CN, NO₂, methyl, ethyl, n-propyl, i-propyl, c-propyl, n-butyl, t-butyl, i-butyl, n-pentyl, 1-methylbutyl, 2-methylbutyl, 3-methylbutyl, 1,1-dimethylpropyl, 1,2-dimethylpropyl, 2,2-dimethylpropyl, methoxyl, ethoxyl, n-propoxyl, i-propoxyl, t-butoxyl, trifluoromethyl, pentafluoroethyl, heptafluoropropyl, heptafluoroisopropyl, difluoromethoxyl, trifluoromethoxyl, pentafluoroethoxyl, methylsulfinyl, trifluoromethylsulfinyl, methylsulfonyl or trifluoromethylsulfonyl;
R₂ is H, methyl, ethyl, n-propyl, i-propyl, n-butyl, i-butyl, t-butyl, n-pentyl, 2-pentyl, neopentyl, isopentyl, 4-methyl-2-pentyl, n-hexyl, monofluoromethyl, difluoromethyl, trifluoromethyl, monochloromethyl, dichloromethyl, trichloromethyl, pentafluoroethyl, heptafluoroisopropyl, cyclopropyl, cyclobutyl, cyclopentyl, perfluorocyclopropyl, perfluorocyclobutyl or perfluorocyclopentyl; and
R₃ is H, F or Cl.

3. The amide compound according to claim 1, wherein, Z₁, Z₂, Z₃, Z₄, and Z₅ are independently of each other H, F, Cl, Br, I, CN, NO₂, methyl, trifluoromethyl, difluoromethoxyl, trifluoromethoxyl, methylsulfonyl or trifluoromethylsulfonyl;
R₁ is H or F;
R₂ is H or methyl;
R₃ is H or Cl; and
W₁ and W₂ are independently of each other O.

4. The amide compound according to claim 1, wherein the amide compound is selected from any one of the compounds below:

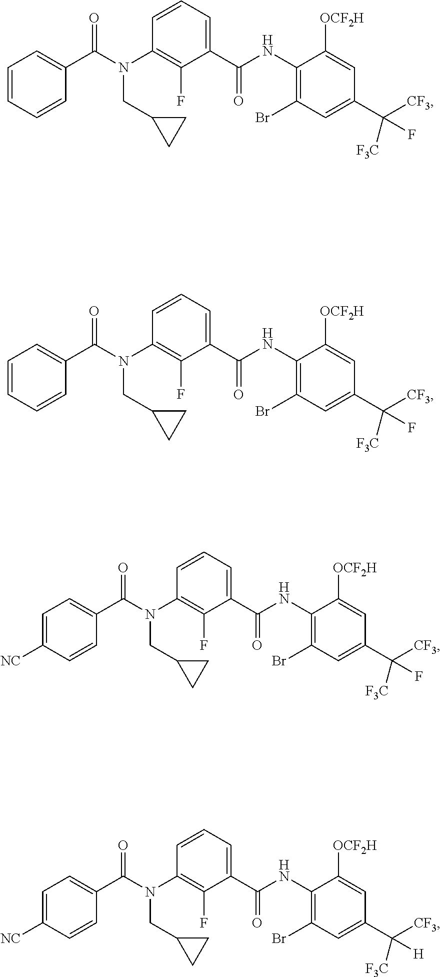

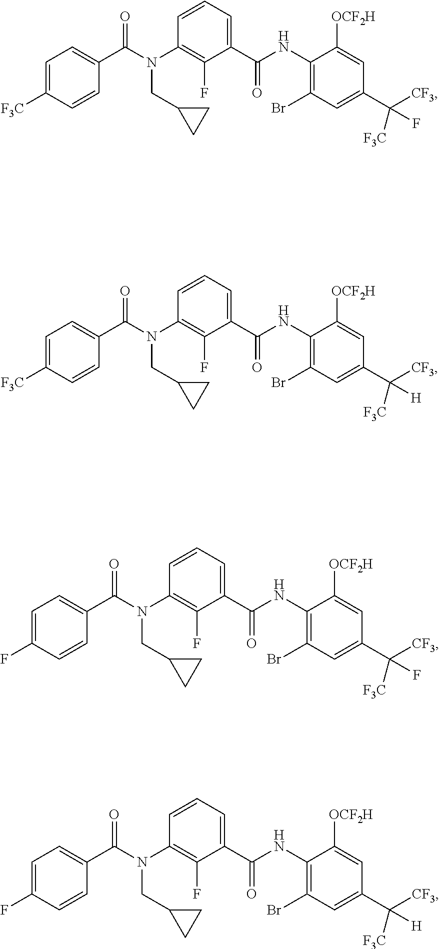

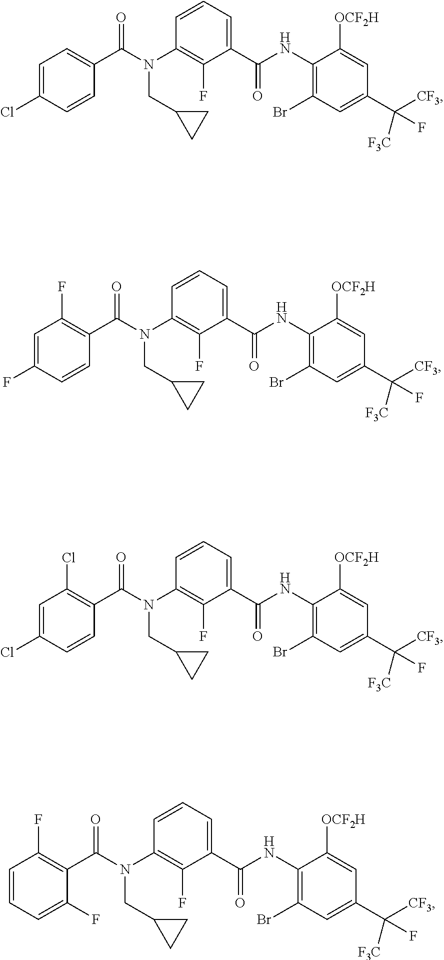

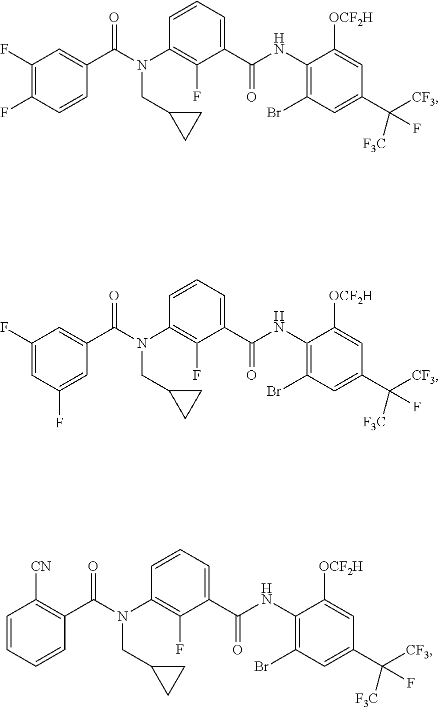

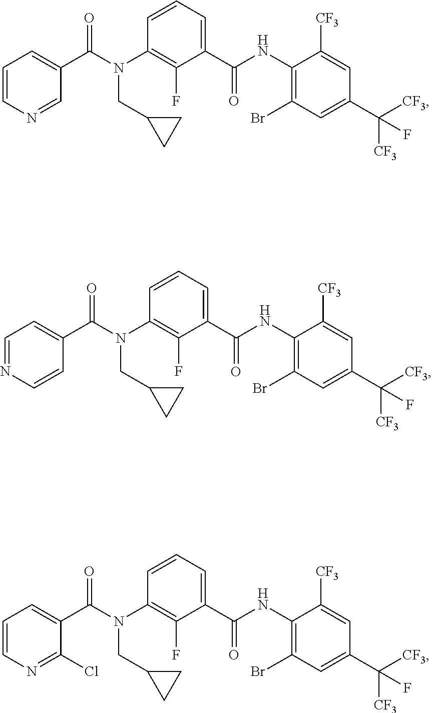 [SMILES: O=C(Nc1c(Br)cc(C(F)(C(F)(F)F)C(F)(F)F)cc1C(F)(F)F)c1cccc(N(CC2CC2)C(=O)c2cccnc2)c1F ; O=C(Nc1c(Br)cc(C(F)(C(F)(F)F)C(F)(F)F)cc1C(F)(F)F)c1cccc(N(CC2CC2)C(=O)c2ccncc2)c1F ; O=C(Nc1c(Br)cc(C(F)(C(F)(F)F)C(F)(F)F)cc1C(F)(F)F)c1cccc(N(CC2CC2)C(=O)c2cccnc2Cl)c1F]

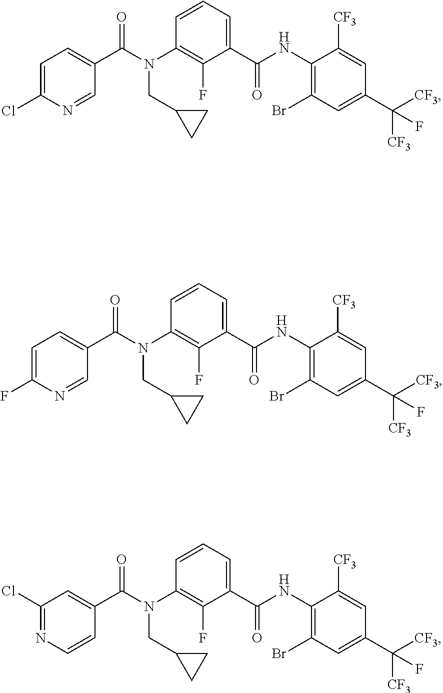 [SMILES: O=C(Nc1c(Br)cc(C(F)(C(F)(F)F)C(F)(F)F)cc1C(F)(F)F)c1cccc(N(CC2CC2)C(=O)c2ccc(Cl)nc2)c1F ; O=C(Nc1c(Br)cc(C(F)(C(F)(F)F)C(F)(F)F)cc1C(F)(F)F)c1cccc(N(CC2CC2)C(=O)c2ccc(F)nc2)c1F ; O=C(Nc1c(Br)cc(C(F)(C(F)(F)F)C(F)(F)F)cc1C(F)(F)F)c1cccc(N(CC2CC2)C(=O)c2ccnc(Cl)c2)c1F]

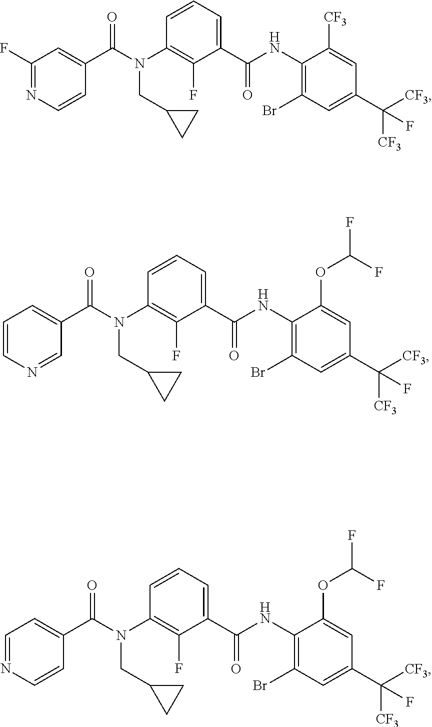 [SMILES: O=C(Nc1c(Br)cc(C(F)(C(F)(F)F)C(F)(F)F)cc1C(F)(F)F)c1cccc(N(CC2CC2)C(=O)c2ccnc(F)c2)c1F ; O=C(Nc1c(Br)cc(C(F)(C(F)(F)F)C(F)(F)F)cc1OC(F)F)c1cccc(N(CC2CC2)C(=O)c2cccnc2)c1F ; O=C(Nc1c(Br)cc(C(F)(C(F)(F)F)C(F)(F)F)cc1OC(F)F)c1cccc(N(CC2CC2)C(=O)c2ccncc2)c1F]

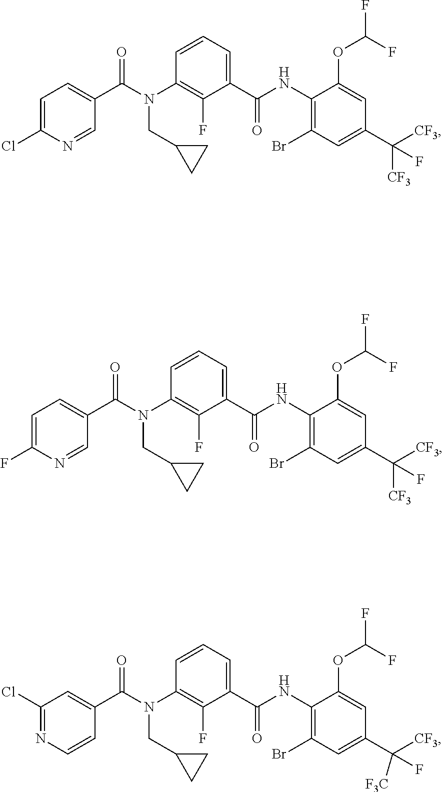 [SMILES: O=C(Nc1c(Br)cc(C(F)(C(F)(F)F)C(F)(F)F)cc1OC(F)F)c1cccc(N(CC2CC2)C(=O)c2ccc(Cl)nc2)c1F ; O=C(Nc1c(Br)cc(C(F)(C(F)(F)F)C(F)(F)F)cc1OC(F)F)c1cccc(N(CC2CC2)C(=O)c2ccc(F)nc2)c1F ; O=C(Nc1c(Br)cc(C(F)(C(F)(F)F)C(F)(F)F)cc1OC(F)F)c1cccc(N(CC2CC2)C(=O)c2ccnc(Cl)c2)c1F]

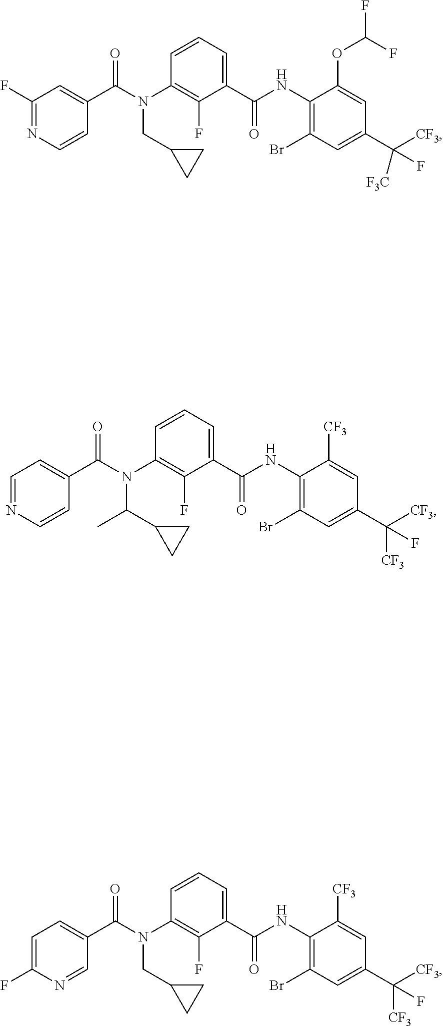 [SMILES: O=C(Nc1c(Br)cc(C(F)(C(F)(F)F)C(F)(F)F)cc1OC(F)F)c1cccc(N(CC2CC2)C(=O)c2ccnc(F)c2)c1F ; CC(C1CC1)N(C(=O)c1ccncc1)c1cccc(C(=O)Nc2c(Br)cc(C(F)(C(F)(F)F)C(F)(F)F)cc2C(F)(F)F)c1F ; O=C(Nc1c(Br)cc(C(F)(C(F)(F)F)C(F)(F)F)cc1C(F)(F)F)c1cccc(N(CC2CC2)C(=O)c2ccc(F)nc2)c1F]

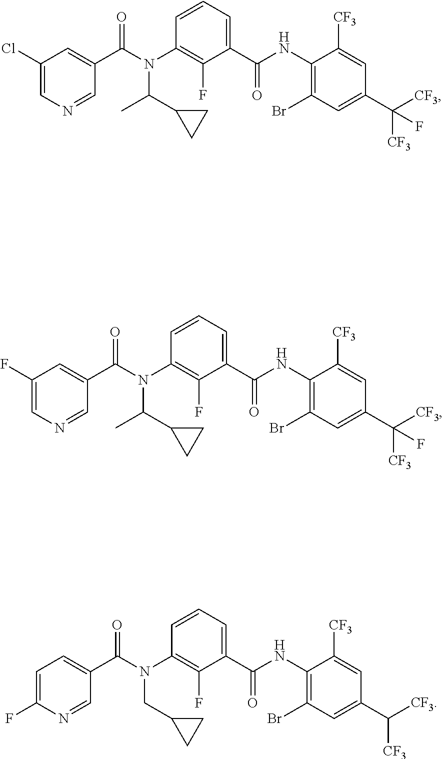 [SMILES: CC(C1CC1)N(C(=O)c1cncc(Cl)c1)c1cccc(C(=O)Nc2c(Br)cc(C(F)(C(F)(F)F)C(F)(F)F)cc2C(F)(F)F)c1F ; CC(C1CC1)N(C(=O)c1cncc(F)c1)c1cccc(C(=O)Nc2c(Br)cc(C(F)(C(F)(F)F)C(F)(F)F)cc2C(F)(F)F)c1F ; O=C(Nc1c(Br)cc(C(C(F)(F)F)C(F)(F)F)cc1C(F)(F)F)c1cccc(N(CC2CC2)C(=O)c2ccc(F)nc2)c1F]

5. The tautomers, enantiomers, diasteromers or salts of the amide compound according to claim 1.

6. An intermediate for preparing the amide compound according to claim 1, wherein the intermediate has a structure as shown in formula XIV: 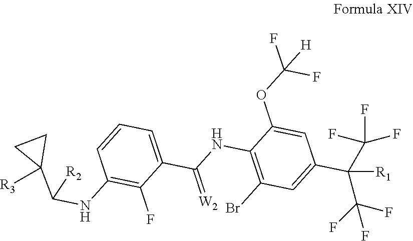
wherein:
R₁ is H or F; R₂ is H, C₁-C₆ haloalkyl, C₃-C₈ cycloalkyl or C₃-C₈ halocycloalkyl;
R₃ is H or halogen; and W₂ are independently of each other O or S.

7. (canceled)

8. An insecticidal composition, characterized in comprising active ingredient(s) and acceptable carrier in agriculture, wherein the active ingredient(s) are the amide compound according to claim 1 or the tautomers, enantiomers, diasteromers or salts thereof.

9. The insecticidal composition according to claim 8, wherein the weight percentage of the active ingredient(s) is 1%-99%.

10. A method for controlling insects, characterized in applying an effective amount of the amide compound according to claim 1, or the tautomers, enantiomers, diasteromers or salts thereof, to pests or their habitat.

11. The method for controlling insects according to claim 10 wherein the effective amount is from 7.5 g/ha to 1000 g/ha.

12. A method for controlling insects, characterized in applying an effective amount of the insecticidal composition of claim 8, to pests or their habitat.
